(19) United States
(12) Patent Application Publication
(10) Pub. No.: US 2024/0079086 A1
(43) Pub. Date: Mar. 7, 2024

(54) EXECUTION AND COMMUNICATION PROTOCOL FOR ALGORITHMIC PROCESSING IN A DIAGNOSTICS SYSTEM

(71) Applicant: TEMPUS LABS, INC., Chicago, IL (US)

(72) Inventors: Charles Jaros, Chicago, IL (US); Nayeem Qadir, Wilmette, IL (US); Isaiah Simpson, Chicago, IL (US); Marc Wielansky, Deerfield, IL (US)

(21) Appl. No.: 17/902,558

(22) Filed: Sep. 2, 2022

(51) Int. Cl. G16B 20/00; G16B 40/20

(52) U.S. Cl. CPC G16B 20/00; G16B 40/20; G16H 50/30

(57) ABSTRACT

The following relates generally to determining genomic biomarkers from biological data (e.g., a read of a nucleic acid, or an image, such as an image of a slide). In some embodiments, a transform orchestrator: (i) receives an order to transform biological data to one or more genomic biomarkers; (ii) selects a transform for deriving each of the received one or more genomic biomarkers; (iii) associates each selected transform with a cloud computing platform; (iv) executes instructions for each selected transform; (v) communicates an operational status of each selected transform; (vi) stores the genomic biomarker output from each selected transform; and (vii) provides a notification of a final operational status of each selected transform.

### BACKGROUND

Diagnostic laboratory tests are commonly used by doctors and other health care professionals to obtain information about whether a subject has a particular condition or disease or whether a treatment of a subject has been effected. Physicians often order tests in order to determine if a particular result has been achieved. For example, if a physician wants to know if a subject has high cholesterol, they may order a test to determine the values of LDL and HDL in the subject's blood. For cancer diagnosis and treatment plans, physicians may order a variety of tests collecting various different types of diagnostic data. These data may include such as radiology scans, molecular profiling, histology slides, and clinical variables. If physicians want to know if a particular sample of tissue is cancerous, they may order a biopsy and have the pathology slides stained and reviewed to determine if a specimen is malignant or benign.

With the rise of precision medicine, systems have been developed to provide diagnostic testing that is subject aware. For example, precision medicine systems may be used to determine genomic biomarkers from collected diagnostic laboratory data for a patient. These biomarkers may predict an individual patient's risk of disease or predict an effectiveness of a potential treatment. Such benefits may even be life saving for a patient. However, current computer systems for intake of diagnostic data are cumbersome and inefficient. Coordination between diagnostic laboratories and precision medicine systems designed to perform biomarker detection is cumbersome and inefficient. As precision medicine systems become more complex and are able to manage greater amounts of diagnostic data, across different collection modalities, the inefficiency in coordination is exacerbated.

There is a need for systems and techniques to better manage operations of precision medicine systems and their coordination with various requesters and data sources.

### SUMMARY

The present application presents systems and methods for applying a transform (e.g., a set of instructions) to biological data (e.g., nucleic acid reads, imaging data, clinical data, molecular data, proteomic data, etc.) to generate an output file (e.g., a genomic biomarker, such as a microsatellite instability, a tumor mutational burden, a variant characterization, a copy number variation, etc.). In examples, the transform, biological data and/or genomic biomarker may relate to one or more disease states, such as an oncological, endocrinological, cardiovascular, and/or neurological disease state. In some examples, a “disease state” refers to a state of disease, such as cancer, cardiology, depression, mental health, diabetes, infectious disease, epilepsy, dermatology, autoimmune diseases, or other diseases. For example, a disease state may indicate the presence or absence of disease in a subject, and may further indicate the severity of the disease. In some implementations, a transform system may deliver information, characteristics, or determinations related to a disease state that may be based on genetic and/or clinical data associated with a patient, specimen and/or organoid. For instance, the transform system may apply a transform to the biological data to determine a genomic biomarker; and, in turn, the genomic biomarker may be used to determine the disease state. Additionally or alternatively, the transform system may apply a transform to the biological data to directly determine the disease state. In various examples, a transform is referred to herein as an insight engine.

In examples, a transform system may receive, from a data source, an order to transform biological data to a genomic biomarker. The transform system may then receive, e.g., from the data source, a workflow, an orchestration platform, a user, or another authoritative source, a selection of a transform for deriving the genomic biomarker; and may then further associate the selected transform system with a cloud computing platform by providing a container image to the cloud computing platform. The cloud computing platform may then execute instructions (e.g., instructions from the container image) for completing the selected transform, and communicate an operational status of the selected transform to the data source, for example through an application programming interface (API) or other connection layer between transforms, requesting services, databases, etc.

In examples, an application program predicts a likelihood of a patient being at a high-risk (e.g., a predicted risk/likelihood exceeding a threshold, such as between 50% and 100%) of one or more of an oncological event, neurological disorder, autoimmune condition, cardiovascular disease, infectious disease, or endocrinological disease. Examples of the oncological event include: a diagnosis of an oncological disease state; a diagnosis of cancer; a response to a therapy; a suitability for a therapy; a suitability for a clinical trial; a progression free survival; a progression of cancer; a metastasis of cancer; and/or an origin of a metastasized tumor. Example determinations related to endocrinological disease include: a diagnosis of an endocrinological disease state; a diagnosis of diabetes; a diagnosis of thyroidism; a diagnosis of an autoimmune disease state; a predicted response to a therapy; a suitability for a therapy; a progression of a disease state; and a suitability for a clinical trial. Example determinations related to neurological disorder may include: a diagnosis of a mental health disease state; a diagnosis of depression; a diagnosis of a mental disorder; a diagnosis of a behavioral disorder; a diagnosis of a personality disorder; a response to a therapy; a suitability for a therapy; a progression of a mental health disease state; and a suitability for a clinical trial. Example determinations related to a cardiac event include: a diagnosis of a cardiovascular disease state; a diagnosis of an arrhythmia; a diagnosis of cardiac arrest; a diagnosis of stroke; a diagnosis of atrial fibrillation; a diagnosis of aortic stenosis; a diagnosis of amyloidosis; a response to a therapy; a suitability for a therapy; a progression of a cardiovascular disease state; and a suitability for a clinical trial.

In accordance with an embodiment, a method for transforming a plurality of nucleic acid reads to one or more genomic biomarkers may be provided. The method may be performed by one or more processors, and the method may comprise: (1) receiving, from a data source, an order to transform the plurality of nucleic acid reads to the one or more genomic biomarkers, wherein the plurality of nucleic acid reads are derived from next generation sequencing of a specimen; (2) receiving a selection of a transform for the order, wherein the transform comprises a configuration, a transform image comprising a plurality of indications of storage locations and a plurality of instructions for completing the transform; (3) associating the selected transform with a cloud computing platform based at least in part on the configuration, the association comprising: (i) providing, to the cloud computing platform, the transform image; (ii) executing, via the cloud computing platform, the plurality of instructions for completing the selected transform; and (iii) loading, via a communication interface, the plurality of nucleic acid reads into a first storage location indicated by the plurality of indications of storage locations; (4) communicating, via the communication interface, at least one communication from the execution between the selected transform and the data source, the at least one communication comprising at least an operational status of the selected transform; (5) storing, via the communication interface, the genomic biomarker output from the selected transform into a second storage location indicated by the plurality of indications of storage locations; and (6) providing a notification, to the data source via the communication interface, of a final operational status of the selected transform based at least in part on the storing the genomic biomarker output from the selected transform.

In some embodiments, the one or more genomic biomarkers are selected from: a microsatellite instability, a tumor mutational burden, a variant characterization, a copy number variation, a fusion, and a presence of a pathology/tissue stain image-derived biomarker. Examples of the stain images include immunohistochemistry (IHC) slide stain images and haematoxylin and eosin (H&E) slide stain images.

In some embodiments, the genomic biomarker comprises microsatellite instability (MSI), and the method further comprises: comparing regions of the genome to at least a portion of the plurality of nucleic acid reads to identify differences and similarities; and reporting the MSI, wherein the MSI comprises a ratio of the identified differences to similarities.

In some embodiments, the method further comprises: accessing, with the transform, an input directory, wherein the input directory is separate from the data source; and writing, with the transform, to an output directory, wherein the output directory is separate from the data source.

In some embodiments, the plurality of nucleic acid reads are in a FASTQ format or a BAMF format.

In some embodiments, the plurality of nucleic acid reads are aligned to a common reference genome.

In some embodiments, the plurality of nucleic acid reads are not aligned to a common reference genome.

In some embodiments, the selection is based on compute requirements of the order to transform the plurality of nucleic acid reads.

In some embodiments, the transforms are associated with the cloud computing platforms based on an available virtual machine (VM) memory size, an available central processing unit (CPU) performance, and a resource quota; and the resource quota comprises a constraint on total compute resources available to: (i) a group of transforms, (ii) a cloud computing system, and/or, (iii) a portion of a cloud computing system.

In some embodiments, the method further comprises: in response to receiving the order to transform the plurality of nucleic acid reads to the one or more genomic biomarkers, determining if the plurality of nucleic acid reads are available to be read; and wherein the selecting of the transforms occurs in response to a determination that the plurality of nucleic acid reads are available to be read. In some embodiments, a workflow orchestrator hosts a “ready state” for the plurality of nucleic acid reads, thereby facilitating determination of if the plurality of nucleic acid reads are available to be read.

In some embodiments, the method further comprises: creating a catalog of transforms for deriving genomic biomarkers by: receiving a plurality of transforms for deriving genomic biomarkers; validating each transform of the received plurality of transforms by determining if there is a problem with each transform; if there is a problem with a transform, returning an error message including an indication of the problem; and updating at least one transform of the plurality of transforms by: receiving an update to the at least one transform; validating the at least one transform by determining if there is a problem with the at least one transform; and if there is a problem with the at least one transform, returning an error message including an indication of the problem with the at least one transform; and wherein the selecting of the transforms comprises selecting the transforms from the created catalog of transforms.

In some embodiments, the notification of the operational status of the data source includes an orchestration error, an execution error, or a timeout error.

In some embodiments, the method further comprises: upon receiving the order to transform the plurality of nucleic acid reads to the one or more genomic biomarkers, determining if the transform should be placed in a high priority queue or a low priority queue; and depending on the determination, placing the order in either the high priority queue or low priority queue; and wherein the executing the plurality of instructions for completing the selected transform occurs by executing instructions in the high priority queue before executing instructions in the low priority queue.

In some embodiments, the configuration is cloud computing platform agnostic.

In some embodiments, the method further comprises predicting, based on the stored genomic biomarker, a likelihood of a patient being at a high-risk of one or more of an oncological event, neurological disorder, autoimmune condition, cardiovascular disease, infectious disease, or endocrinological disease.

In some embodiments, the method further comprises predicting, based on the stored genomic biomarker, one or more of: an onset of an oncological disease state; an onset of cancer; a response to a cancer therapy; a suitability for a cancer therapy; a suitability for a cancer clinical trial; a progression free cancer survival; a progression of cancer; a metastasis of cancer; and/or an origin of a metastasized tumor.

In some embodiments, the method further comprises predicting, based on the stored genomic biomarker, one or more of: an onset of an endocrinological disease state; an onset of diabetes; an onset of thyroidism; an onset of an autoimmune disease state; a response to an endocrinological therapy; a suitability for an endocrinological therapy; a progression of an endocrinological disease state; and/or a suitability for an endocrinological clinical trial.

In some embodiments, the method further comprises predicting, based on the stored genomic biomarker, one or more of: an onset of a mental health disease state; an onset of depression; an onset of a mental disorder; an onset of a behavioral disorder; an onset of a personality disorder; a response to a neurological therapy; a suitability for a neurological therapy; a progression of a mental health disease state; and/or a suitability for a neurological clinical trial.

In some embodiments, the method further comprises predicting, based on the stored genomic biomarker, one or more of: an onset of a cardiovascular disease state; an onset of an arrhythmia; an onset of cardiac arrest; an onset of stroke; an onset of atrial fibrillation; an onset of aortic stenosis; an onset of amyloidosis; a response to a cardiovascular therapy; a suitability for a cardiovascular therapy; a progression of a cardiovascular disease state; and/or a suitability for a cardiovascular clinical trial.

In some embodiments, an “onset” includes an active onset of the condition, as well as future likelihood above a predetermined likelihood threshold of the condition occurring within a period of time.

In accordance with another embodiment, a computer system for transforming a plurality of nucleic acid reads to one or more genomic biomarkers may be provided. The computer system may comprise one or more processors configured to: (1) receive, from a data source, an order to transform the plurality of nucleic acid reads to the one or more genomic biomarkers, wherein the plurality of nucleic acid reads are derived from next generation sequencing of a specimen; (2) receive a selection of a transform for the order, wherein the transform comprises a configuration, a transform image comprising a plurality of indications of storage locations and a plurality of instructions for completing the transform; (3) associate the selected transform with a cloud computing platform based at least in part on the configuration, the association comprising: (i) providing, to the cloud computing platform, the transform image; (ii) executing, via the cloud computing platform, the plurality of instructions for completing the selected transform; and (iii) loading, via a communication interface, the plurality of nucleic acid reads into a first storage location indicated by the plurality of indications of storage locations; (4) communicate, via the communication interface, at least one communication from the execution between the selected transform and the data source, the at least one communication comprising at least an operational status of the selected transform; (5) store, via the communication interface, the genomic biomarker output from the selected transform into a second storage location indicated by the plurality of indications of storage locations; and (6) provide a notification, to the data source via the communication interface, a final operational status of the selected transform based at least in part on the storing the genomic biomarker output from the selected transform.

In some embodiments, the one or more processors are further configured to: in response to receiving the order to transform the plurality of nucleic acid reads to the one or more genomic biomarkers, determine if the plurality of nucleic acid reads are in a ready state; and make the selection of the transforms in response to a determination that the plurality of nucleic acid reads are in the ready state.

In some embodiments, the one or more processors are further configured to: create a catalog of transforms for deriving genomic biomarkers by: receiving a plurality of transforms for deriving genomic biomarkers; validating each transform of the received plurality of transforms by determining if there is a problem with each transform; if there is a problem with a transform, returning an error message including an indication of the problem; and updating at least one transform of the plurality of transforms by: receiving an update to the at least one transform; validating the at least one transform by determining if there is a problem with the at least one transform; and if there is a problem with the at least one transform, returning an error message including an indication of the problem with the at least one transform; and wherein the selecting of the transforms comprises selecting the transforms from the created catalog of transforms.

In accordance with yet another embodiment, a computing device for transforming a plurality of nucleic acid reads to one or more genomic biomarkers may be provided. The computing device may comprise: one or more processors; and one or more memories coupled to the one or more processors. The one or more memories including computer executable instructions stored therein that, when executed by the one or more processors, may cause the one or more processors to: (1) receive, from a data source, an order to transform the plurality of nucleic acid reads to the one or more genomic biomarkers, wherein the plurality of nucleic acid reads are derived from next generation sequencing of a specimen; (2) receive a selection of a transform for the order, wherein the transform comprises a configuration, a transform image comprising a plurality of indications of storage locations and a plurality of instructions for completing the transform; (3) associate the selected transform with a cloud computing platform based at least in part on the configuration, the association comprising: (i) providing, to the cloud computing platform, the transform image; (ii) executing, via the cloud computing platform, the plurality of instructions for completing the selected transform; and (iii) loading, via a communication interface, the plurality of nucleic acid reads into a first storage location indicated by the plurality of indications of storage locations; (4) communicate, via the communication interface, at least one communication from the execution between the selected transform and the data source, the at least one communication comprising at least an operational status of the selected transform; (5) store, via the communication interface, the genomic biomarker output from the selected transform into a second storage location indicated by the plurality of indications of storage locations; and (6) provide a notification, to the data source via the communication interface, a final operational status of the selected transform based at least in part on the storing the genomic biomarker output from the selected transform.

In some embodiments, the notification of the operational status of the data source includes an orchestration error, an execution error, or a timeout error.

In some embodiments, the one or more memories including computer executable instructions stored therein that, when executed by the one or more processors, further cause the one or more processors to: create a catalog of transforms for deriving genomic biomarkers by: receiving a plurality of transforms for deriving genomic biomarkers; validating each transform of the received plurality of transforms by determining if there is a problem with each transform; if there is a problem with a transform, returning an error message including an indication of the problem; and updating at least one transform of the plurality of transforms by: receiving an update to the at least one transform; validating the at least one transform by determining if there is a problem with the at least one transform; and if there is a problem with the at least one transform, returning an error message including an indication of the problem with the at least one transform; and wherein the selecting of the transforms comprises selecting the transforms from the created catalog of transforms.

### BRIEF DESCRIPTION OF THE DRAWINGS

FIG. 1 illustrates an example precision medicine system architecture for use in determining a genomic biomarker in response to a request from a data source, in accordance with an example.

FIG. 2A illustrates an example of a transform system of the precision medicine system of FIG. 1 and having a transform orchestrator interfacing with data sources, plurality of storage locations, and plurality of cloud computing platforms, in accordance with an example.

FIG. 2B is a schematic of an example transform orchestrator, and example processes of the transform orchestrator intaking transforms and executing transforms, in accordance with an example.

FIG. 3A is a schematic of an example of a container for implementing one or more transforms of the transform orchestrator, in accordance with an example.

FIG. 3B is a schematic of an example of a docker container having a docker layer and a series of containers, in accordance with an example.

FIG. 3C is a schematic of an example transform, in accordance with an example.

FIG. 4 is a flowchart of an example method of transforming biological data to one or more genomic biomarkers using the transform system and transform of FIGS. 2A & 2B, in accordance with an example.

FIG. 5 is a flowchart of an example method relating to the transform orchestrator receiving an order and biological data from a data source server, in accordance with an example.

FIG. 6 illustrates an example method of creating a catalog of transforms, in accordance with an example.

FIG. 7 illustrates an example transform system as may be used to implement the transform system of FIG. 2A, in accordance with an example.

FIG. 8 illustrates an example transform signal diagram, in accordance with an example.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

In various embodiments, systems and methods are disclosed for facilitating intake, processing, and storage of patient diagnostic data (including biological data such as pathology imaging data, radiology imaging data, clinical data, molecular data, proteomic data, etc.) and requests for precision medicine system access. The systems and methods control access to different transforms of a precision medicine, including, in some examples, different trained machine learning transforms each capable of generating one or more genomic biomarkers in response to the diagnostic data. The systems and methods control reporting of such biomarker determinations to the requester.

Precision medicine systems may contain many different computational processes related to biomarker determination, including, in various examples herein, numerous different transforms each built to generate one or more biomarker determinations from received diagnostic data. Conventionally, development teams of engineers, coders, and scientists are responsible for all aspects of managing their respective computational process including but not limited to scheduling, data orchestration, tracing and monitoring for their respective processing pipelines. That results in limitations, such as repeated build of similar but different capabilities (that are difficult to reuse) with varying degrees of operational readiness. Further, there is considerable overhead associated with maintaining precision medicine systems that have high variance in implementation hardiness and capabilities. The techniques described herein create a unified structure for implementation across the different teams, thereby guarding against mistakes, and enabling more efficient systems.

As used herein references to a “transform” refers to a set of instructions that operate on an input file (e.g., including diagnostic data such as biological data such as imaging data, clinical data, molecular data, proteomic data, etc.) to determine an output file (e.g., including a genomic biomarker). In some implementations, transforms receive and consume biological or other data and generate a biomarker that relates to one or more disease states. A transform may generate (and a transform system may deliver) information, characteristics, or determinations related to a disease state that may be based on genetic and/or clinical data associated with a patient, specimen and/or organoid. For example, each transform of a transform system may apply a different transform to biological data to determine a different genomic biomarker; and, in turn, one or more of the genomic biomarkers may be used to determine the disease state. Additionally or alternatively, the transform may apply a transform to the biological data to directly determine the disease state. More generally, transforms may apply a transform to biological data and/or other received data for a subject and determine one or more biomarkers, predicted state, and/or risk scores indicating a likelihood of a medical event occurrence. For convenience sake, in various examples herein, reference is made to a determination of biomarker by a transform, and such references are intended to include determination of a biomarker, predicted state, and/or risk factor. In various examples, a transform is referred to herein as an insight engine.

As used herein references to a transform includes any suitable executable process capable of receiving and consuming biological or other data and generating a biomarker, a predict state, and/or a risk score, including by way of example, and not limitation, the following.

An example transform includes an application configured to determine microsatellite instability (MSI) from analysis of genomic sequencing reads from a tumor specimen using an MSI engine as disclosed, for example, in U.S. Patent Publication No. 2020/0118644, titled “Microsatellite Instability Determination System and Related Methods.” For example, a transform may determine MSI directly from microsatellite region mappings for specific loci in the genome. For example, a transform may receive, in corresponding data files, a locus for a plurality of MSI loci and genomic sequencing reads for a tumor specimen, and map a first plurality of genomic sequencing reads from a tumor specimen to the locus and map a second plurality of genomic sequencing reads from a matched-normal specimen to the locus. The transform may compare these mappings and determine the likelihood of microsatellite instability based on the comparison, from which a report is generated indicating the determined likelihood of microsatellite instability.

Another example transform includes an application configured to determine tumor mutational burden (TMB) using a TMB engine as disclosed, for example, in U.S. Patent Publication No. 2020/0258601, titled “Targeted-Panel Tumor Mutational Burden Calculation Systems and Methods.” For example, a transform may determine TMB by receiving next generation sequencing data for a patient's germline specimen in a data file and identifying sequences of nucleotides in the germline specimen using a targeted-panel, receiving next generation sequencing data for a patient's somatic specimen and identifying sequences of nucleotides in the somatic specimen using the targeted-panel, and calculating a TMB status from mutations in the germline sequencing results and from mutations in the somatic sequencing results, when certain passing threshold results are overcome, and generating a reporting indicating the determined TMB status.

Another example transform includes an application configured to determine variant characterization based on biological data captured from a liquid biopsy assay as disclosed, for example, in U.S. patent application Ser. No. 17/179,086, titled “Methods And Systems For Dynamic Variant Thresholding In A Liquid Biopsy Assay”, U.S. patent application Ser. No. 17/179,267, titled “Estimation Of Circulating Tumor Fraction Using Off-Target Reads Of Targeted-Panel Sequencing”, and U.S. patent application Ser. No. 17/179,279, titled “Methods And Systems For Refining Copy Number Variation In A Liquid Biopsy Assay.” For example, a transform may determine a variant (e.g., variant alleles) by receiving sequencing reads, from a data file, for each cell-free DNA fragment in a liquid biopsy sample, aligning each sequencing read to a reference sequence and identifying candidate somatic sequence variants, for each candidate variant, determining a variant allele fragment count and a respective locus fragment count, and comparing the allele fragment count for each candidate against a dynamic variant count threshold to reject or not reject various candidate variants, and generating a reporting indicating the determined variants.

Another example transform includes an application configured to determine gene fusions using a programmed-death ligand 1 (PD-L1) status engine as disclosed, for example, in U.S. Patent Publication No. 2020/0395097, titled “A Pan-Cancer Model to Predict The PD-L1 Status of a Cancer Cell Sample Using RNA Expression Data and Other Patient Data” and in U.S. Pat. No. 10,957,041, titled “Determining Biomarkers from Histopathology Slide Images.” For example, a transform may determine PD-L1 status by receiving, from a data file, unlabeled gene expression data for a sample and aligning that unlabeled expression data to a set of labeled expression data according to a trained PD-L1 predictive model to identify a PD-L1 expression status for the sample, and generating a reporting indicating the PD-L1 expression status.

Another example transform includes an application configured to determine the presence of an H&E-derived biomarker or IHC-derived biomarker using an image-based biomarker prediction system as disclosed in U.S. Pat. No. 10,957,041, titled “Determining Biomarkers from Histopathology Slide Images.” For example, a transform may determine a pathology/tissue stain image-derived biomarker by receiving digital images of a stained tissue sample, separating the digital image into a plurality of tiles, applying the tiles to a deep learning framework having one or more trained biomarker classification models each trained to classify a different biomarker, predicting a biomarker classification for each of the tiles, determining from those predicted classifications a predicted presence of at least one biomarker, and generating a report of the predicted at least one biomarker.

Another example transform includes an application configured to determine the presence of a mental disorder using a system generating information on a patient diagnoses with a psychiatric illness as disclosed in U.S. Publication No. 2021/0012882, titled “Data-based mental disorder research and treatment systems and methods.” For example, a transform may receive molecular data, in a data file, from a multi-gene panel sequencing reaction from a sample, align the molecular data to a reference sequence, receiving clinical data identifying medications and diagnoses for a patient, using a therapy engine to generate a report from the molecular and clinical data where the report includes a phenotype associated with molecular data and drugs and drug classification. That report may be generated and reported to a user. Further the therapy engine can be updated with subsequent clinical data obtained for the patient.

Another example transform includes an application configured to predict the likelihood that a patient will suffer from atrial fibrillation as disclosed in U.S. Publication No. 2021/0076960, titled “ECG based future atrial fibrillation predictor systems and methods.” For example, a transform may receive patient electrocardiogram data, in a data file, and an electrocardiogram configuration, in a data file, receive patient demographic data such as age and sex, and providing the received data to a trained model trained to generate a risk score, receiving from the trained model a risk score indicative of a likelihood the patient will suffer from a condition within a predetermined period of time, and generating a report of the risk score.

An example transform configured in the laboratory diagnostic space is disclosed in U.S. Publication 2021/0118559, titled “artificial intelligence assisted precision medicine enhancements to standardized laboratory diagnostic testing.” For example, a transform may: (i) receive patient information (e.g., from a profile of the patient); (ii) receive a lab diagnostic (e.g., an initial result of an examination of a specimen or an image associated with the patient); and (iii) output an adjustment to the lab diagnostic based on the patient information.

In this regard, it should be understood that using biological and other data to determine a genomic biomarker, predicted state, or risk score has many health benefits for a patient. For instance, a genomic biomarker, such as a microsatellite instability, a tumor mutational burden, a variant characterization, a copy number variation, a fusion, or a presence of an Immunohistochemistry (IHC)-derived biomarker, may be determined; and this biomarker determination may be further used to predict a patient's risk of disease, predict an effectiveness of a potential treatment, or identify a recommended subset of available treatment options.

### Example System

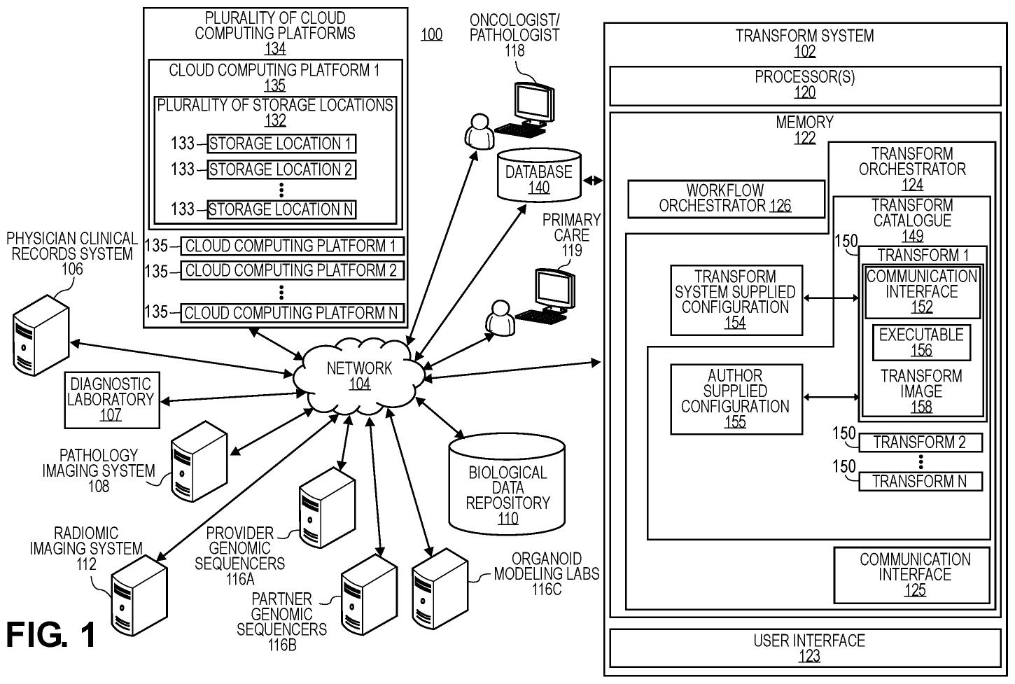

To this end, FIG. 1 illustrates an example precision medicine architecture 100 that may be used for determining a genomic biomarker by applying one or a number of transforms to biological data received from the data sources and/or stored on the transform system.

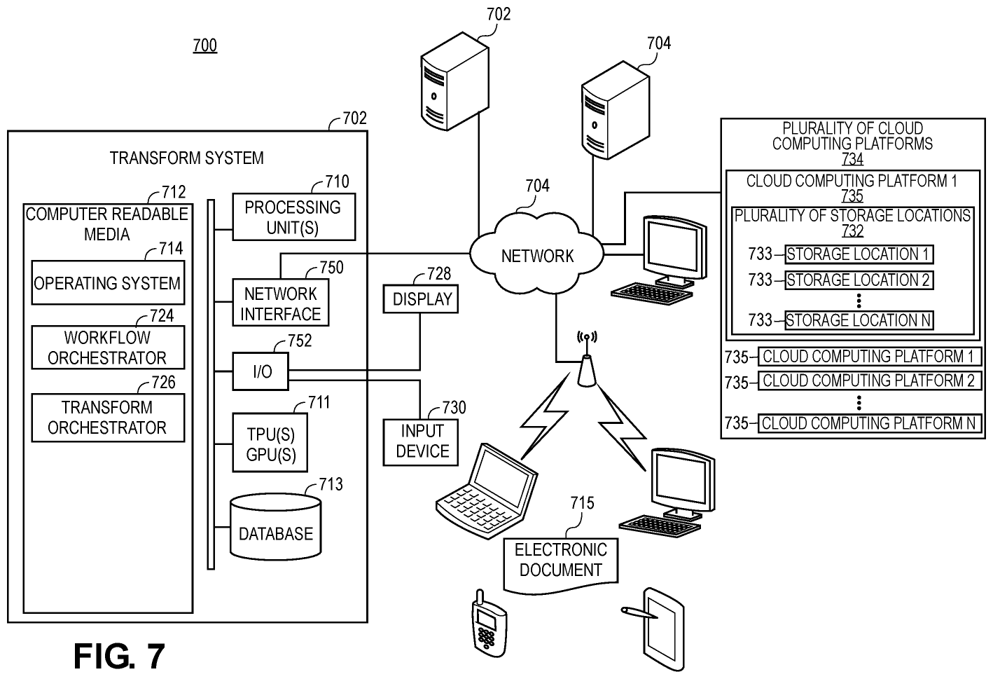

The architecture 100 may include a transform system 102 configured to communicate, e.g., via a network 104 (which may be a wired or wireless network, as discussed further below in reference to FIG. 7), with a variety of data sources. Example data sources include: a physician clinical records system 106, diagnostic laboratory 107, a pathology imaging system 108, a biological data repository 110, a radiomic imaging system 112, provider genomic sequencers 116A, partner genomic sequencers 1168, organoid modeling labs 116C, pathology lab/oncology system 118, and primary care provider computer system 119.

The physician clinical records system 106 may include, for example, record systems of a physician's office, or record systems of any clinician's office. For instance, a physician's office or clinician's office may store biological data of a patient which it is desired to derive a genomic biomarker from.

The diagnostic laboratory 107 may be a laboratory that processes patient data (e.g., biological data), and/or that makes diagnosis based on patient data. For example, the diagnostic laboratory 107 may make diagnosis based upon slide images, H&E stain images, IHC images, radiology images, etc.

The pathology imaging system 108 may be, for example, a system that gathers and/or stores images of tissue slides (e.g., such as haematoxylin and eosin (H&E) stain images, immunohistochemistry (IHC) images, or other slide images).

The biological data repository 110 may be, for example, a general purpose database of biological data. For instance, biological data repository 110 may be a database of a hospital, a government entity, etc. Such databases may hold any kind of data. For example, the data may include biological data, such as nucleic acid reads, imaging data, clinical data, molecular data, proteomic data, etc. In another example, the data may include patient medical information, such as a medical history of a patient corresponding to biological data.

The radiomic imaging system 112 may be, for example, a system that extracts features from medical images. For instance, the radiomic imaging system 112 may extract features from any kind of radiological images, such as computed tomography (CT) images, magnetic resonance imaging (MRI) images, positron emission tomography (PET)/CT images, PET/MR images, etc.

The provider genomic sequencers 116A may be any kind of genomic sequencers (e.g., sequences that determine nucleic acid reads). For instance, the provider genomic sequencers 116A may directly acquire a sample from a patient and determine nucleic acid reads from the acquired sample.

The partner genomic sequencers 1168 may be any kind of genomic sequencers (e.g., sequences that determine nucleic acid reads). For instance, the partner genomic sequencers 1168 may be an entity that receives digital data (e.g., possibly from the provider genomic sequencers 116A, or any other entity), and then processes the digital data to determine a nucleic acid read. In another example, the partner genomic sequencers 1168 may correspond to individual patients (e.g., a sequencer may be in the home of an individual patient). In yet another example, the partner genomic sequencers 1168 may be external sequencers, such as sequencers from a partner laboratory, institution, or personal user.

The organoid modeling labs 116C may be, for example, a lab that participates in the development of an organoid (e.g., an artificially grown mass of cells or tissue resembling an organ). For example, the organoid modeling labs 116C may process medical data of a patient to create a model of an organoid for the patient.

The pathology lab/oncology system 118 may be, for example, a lab that acquires samples from a patient for the purpose of cancer and/or tumor diagnosis.

The primary care provider computer system 119 may be, for example, a computer system of a primary care provider to a patient for whom it is desired to determine a genomic biomarker for.

The data sources may generate and/or store, inter alia, biological data. Examples of the biological data include nucleic acid reads (e.g., reads of deoxyribonucleic acid (DNA) or ribonucleic acid (RNA)), and images (e.g., pathology slide images, haematoxylin and eosin (H&E) stain images, IHC stain images, etc.).

Further, the data sources 106-119 may request access to resources of the precision medicine architecture 100, in particular requests for analysis of respective biological data and determination of resulting biomarkers determined from the same. Toward this end, the data sources (e.g., physician clinical records system 106, pathology imaging system 108, biological data repository 110, radiomic imaging system 112, provider genomic sequencers 116A, partner genomic sequencers 1168, organoid modeling labs 116C, pathology lab/oncology system 118, and/or primary care provider computer system 119) may each respectively include one or more processors such as one or more microprocessors, controllers, and/or any other suitable type of processor. The data sources may each respectively further include a memory (e.g., volatile memory, non-volatile memory) accessible by the respective one or more processors (e.g., via a memory controller). The respective one or more processors may each interact with the respective memories to obtain, for example, computer-readable instructions stored in the respective memories. Additionally or alternatively, computer-readable instructions may be stored on one or more removable media (e.g., a compact disc, a digital versatile disc, removable flash memory, etc.) that may be coupled to the data sources servers to provide access to the computer-readable instructions stored thereon. The data sources 106-119 may include user interfaces to allow users, such as medical professionals, to request access to resources of the precision medicine architecture 100 and to display reports of outputs received from those resources.

Resources of the precision medicine architecture 100 may be stored on or otherwise accessed by the transform system 102, which may include one or more processors 120 such as one or more microprocessors, controllers, and/or any other suitable type of processor. The transform system 102 may further include a memory 122 (e.g., volatile memory, non-volatile memory) accessible by the one or more processors 120, (e.g., via a memory controller). Additionally, the transform system 102 may include a user interface 123.

The one or more processors 120 may interact with the memory 122 to obtain, for example, computer-readable instructions stored in the memory 122. Additionally or alternatively, computer-readable instructions may be stored on one or more removable media (e.g., a compact disc, a digital versatile disc, removable flash memory, etc.) that may be coupled to the transform system 102 to provide access to the computer-readable instructions stored thereon. In particular, the computer-readable instructions stored on the memory 122 may include instructions for executing various applications.

Plurality of cloud computing platforms 131 may also be connected to the network 104, and may include cloud computing platforms 135. The cloud computing platforms 135 may include a plurality of storage locations 132 including individual storage locations 133. It should be understood that a cloud computing platforms 135 may be any hardware, software, or combination thereof used to host an application or service. In one example, a cloud computing platform 135 includes hardware, an OS, and coordinating programs using an instruction set for a specific microprocessor. In some embodiments, a cloud computing platform 135 may have different abstraction levels, such as a computer architecture, an OS, and/or a runtime library. It should further be understood that certain algorithms may be platform-specific (e.g., it was developed to run on a certain platform), cross-platform, or platform-agnostic. In some embodiments, the transform system 102 also builds database 140 (e.g., a database of genomic biomarkers of patients).

Furthermore, in some embodiments, data may be sent from any entity connected to the network 104 to the database 140. In one example, one or more entities connected to network 104 may each provide various types of data for a patient, clinical, imaging, genomic, demographic, etc. Such networking advantageously increases the amount of data available to the database 140, and further advantageously allows additional types of data to be available to the database 140. As this example illustrates, the database 140 may store any kind of data regardless of whether a transform generates it or whether the data is obtained from other sources. Further, in some embodiments, any consumer of data may access data provisioned from database 140, for example, through an authorization access structure that authenticates a user or user system and grants access to certain data within the database 140.

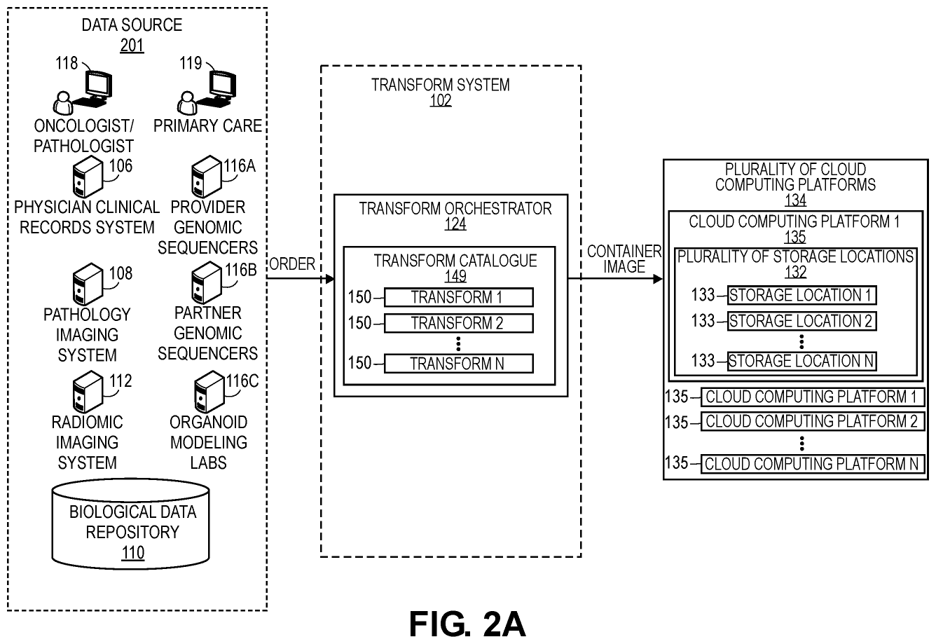

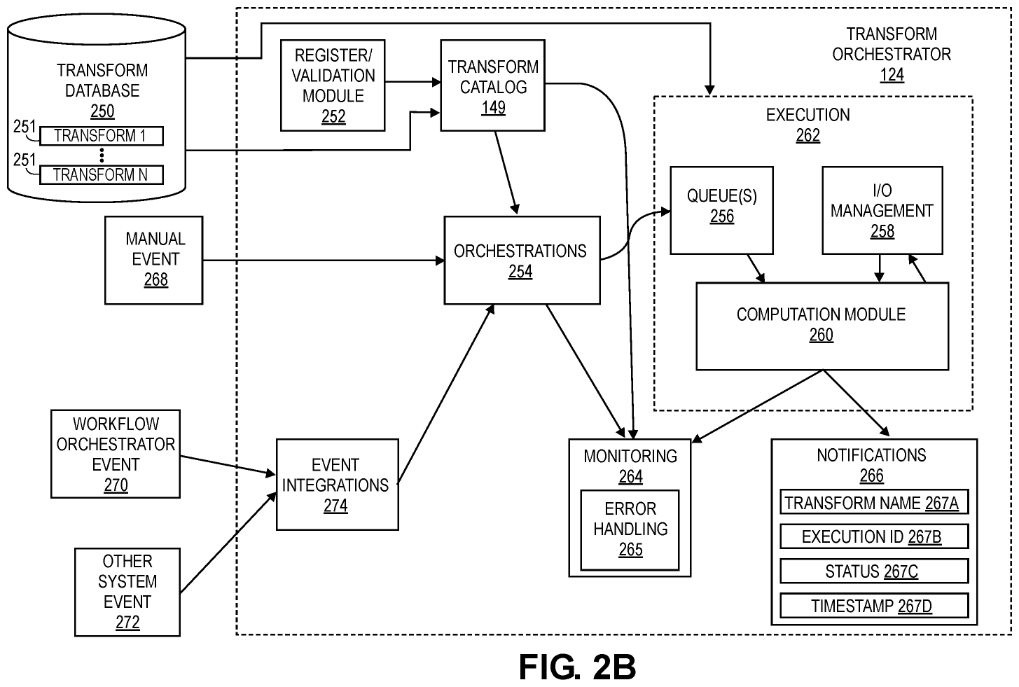

The memory 122 may further include transform orchestrator 124, which may be used to store and apply transforms 150. To this end, the transform orchestrator 124 may build a transform catalog 149 (e.g., by receiving, validating, and storing transforms 150). Example implementations of the transform orchestrator 124, the transform catalog 149, and their functions will be further detailed elsewhere herein, particularly with respect to FIG. 2A.

The transform orchestrator 124 may further include a communication interface 125, for example, to send and/or receive biological data, such as biological data from a data source. Furthermore, although the example system 100 illustrates the transform orchestrator 124 as part of the transform system 102, in some embodiments, the transform orchestrator 124 exists outside of the transform system 102 as its own service that provides orchestration to the transform system 102.

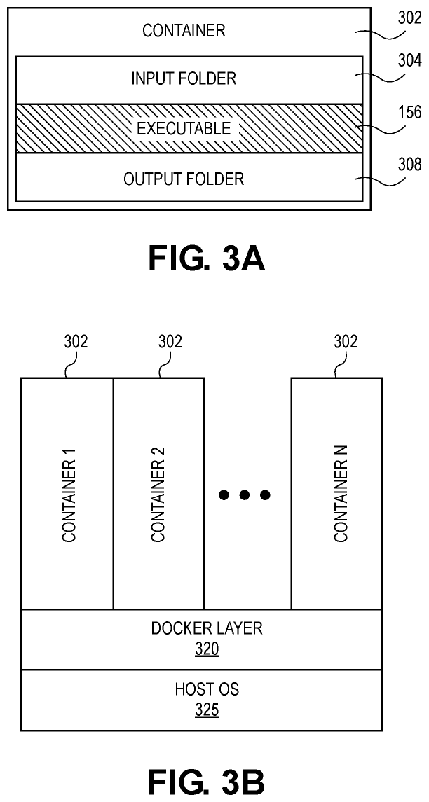

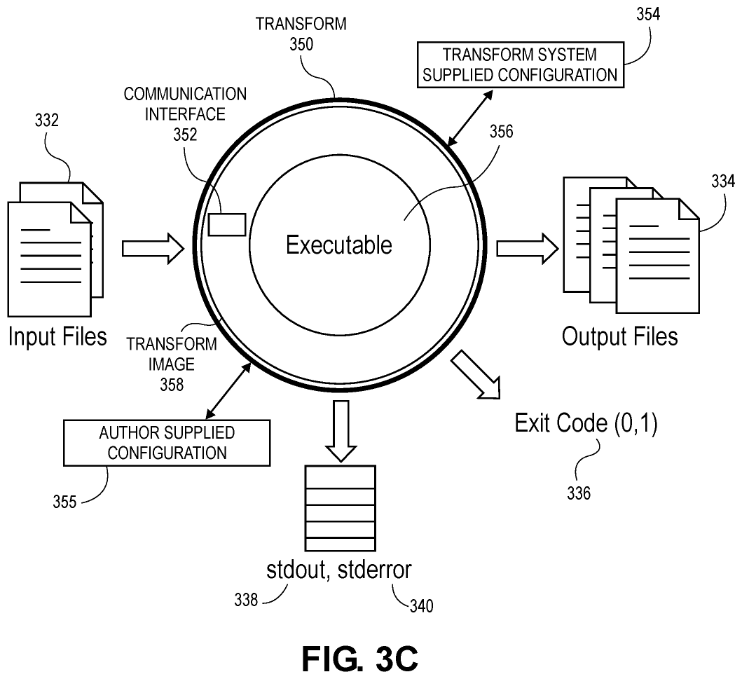

Each transform 150 may include a transform image 158, which may comprise a plurality of indications of storage locations. Additionally or alternatively, the transform image 158 may comprise a full or partial uniform resource locator (URL) of a custom disk image, which may contain the data to be mounted to a container. Examples of these components of the transform 150 will be further detailed elsewhere herein, particularly with respect to FIG. 3C.

In various examples, each transform image 158 may comprise a communication interface 152 (e.g., for sending and/or receiving data, such as biological data, from a data source, that the transform 150 will use). Also illustrated is a transform system supplied configuration 154 (e.g., including transform registration data, transform metadata, information for identifying or fetching inputs, information for storing outputs, trigger definitions to cause the transform to begin, etc.). The transform system supplied configuration 154, in some implementations, is generated at the time the transform 150 is executed. In this regard, to execute the transform, the transform image 158 may further include an executable 156 (e.g., an executable code portion of the transform 150, such as a plurality of instructions for completing the transform 150). In some embodiments, the transform image 158 is required to be compatible with particular rules. For example, the transform image 158 may be validated such that that the transform image 158: is based on an OS with proper support (e.g., supports an apt-get); supports certain commands (e.g., supports an “unshare” command); and/or does not already contain a particular directory.

Some embodiments also include transform author supplied configuration 155, which may include details of the transform 150. Some examples of information that the author supplied configuration 155 may include are: (i) a unique name of the transform; (ii) an indication of the author or owner; (iii) a specification of the author's executable/container image containing their executable; (iv) specification of the input and output parameters; (v) input and output parameters that may indicate persistent volumes/storage locations; (vi) specification of the amount and type of compute resources required to run the executable (CPU, memory, disk space); and/or (vii) specification of signals from the workflow orchestrator 126 which: (a) cause the transform to be executed, and (b) optionally include instructions on how to identify input parameters from data stored within the workflow orchestrator 126.

In some embodiments, the transform orchestrator 124 is able to choose which cloud computing platform 135 to utilize when a transform 150 is triggered by an actor (e.g., a person, a build system, etc.). Depending on which cloud computing platform 135 is chosen, a configuration may be generated by the transform orchestrator 124 which allows the transform 150 to function correctly within the target cloud computing platform 135, including storage locations 133, access tokens to utilize cloud provider services, etc.

In some embodiments, the transform system 102 determines a genomic biomarker by applying the transform 150 to the biological data. In this regard, one or more of the data sources may send a transform order request (e.g., to access precision medicine resources) to the transform system 102 (e.g., an order request to determine the genomic biomarker from the biological data). In some embodiments, along with the order, the data sources also send the biological data itself.

In some example implementations, to execute the transform 150 to determine the genomic biomarker, the transform system 102 may associate transforms with one or more of a plurality of cloud computing platforms 134. This association may include loading the received biological data into a first storage location 133 of the plurality of storage locations 132. This first storage location 133 may have been indicated by the transform image 158. The association may further comprise providing the transform image 158 to a cloud computing platform 135 of the plurality of cloud computing platforms 134. While the example of FIG. 1 illustrates the plurality of cloud computing platforms 134 as directly part of the transform system 102, in some embodiments, the plurality of cloud computing platforms 134 may instead be communicatively coupled to the transform system 102, for example through the network 104. The cloud computing platform 135 may then execute a plurality of instructions (e.g., the executable 156) of the transform 150. Communications may be sent (e.g., via communication interface 125 and/or the communication interface 152) back to the data source to update the data source of the operational status of the transform 150. However, it should be understood that the transform 150 itself does not communicate directly outside of the transform orchestrator 124. For example, the transform 150 may communicate with the transform orchestrator 124, which then determines if a communication should be acted upon within the transform orchestrator 124, or should be relayed externally (e.g., to the plurality of cloud computing platforms 134, etc.). The execution of the plurality of instructions may produce the genomic biomarker output, which may then be stored in a second storage location 133 of the plurality of storage locations 132. Subsequently, a notification may be provided (e.g., via the communication interface) to the data source indicating a final operational status of the transform 150; for example, the notification of the final operational status may be provided based on the genomic biomarker output being stored to the second storage location 133.

Furthermore, multiple storage locations (e.g., the first and second storage locations) may be accessible by different actors. Thus, advantageously, being able to write to a second storage location allows the transform to write data such that is accessible by actors that do not have access to the first. Moreover, data can be received upstream in a pipeline (e.g., of storage locations) and deposited downstream in the pipeline. However, it should be understood that the first and second storage locations may be the same or different storage locations.

In addition, it may be noted that although the example of FIG. 1 illustrates only one transform system, any number of transform systems may be used. In this regard, the memory 122, transform orchestrator 124, workflow orchestrator 126, plurality of storage locations 132 and plurality of cloud computing platforms 134 are not limited to only one transform system. That is, the memory 122, transform orchestrator 124, workflow orchestrator 126, plurality of storage locations 132 and plurality of cloud computing platforms 134 may be distributed across multiple transform systems and/or processors.

In some embodiments, the workflow orchestrator 126 coordinates workflows, which are one or more sequences that are implemented within a precision medicine system and that may include steps through an order ingestion to generate a report. As such, as discussed further below, the workflow orchestrator 126 may detect errors in workflows identified from such coordination. In some embodiments, the workflow orchestrator is separate, rather than part of, the transform system 102. In some such embodiments, the workflow orchestrator 126 communicates with the transform system 102 through the network 104. Furthermore, in some such embodiments where the workflow orchestrator 126 is separate from the transform system 102, there may be multiple transform orchestrators 124 and transforms 150; and the workflow orchestrator 126 may control all of the transform orchestrators 124 and transforms 150. In addition, there may be multiple external workflow orchestrators 126, which may control any or all transform orchestrator(s) 124 and/or transforms 150.

FIG. 2A illustrates an example of a transform orchestrator 124 in relation to a data source 201, plurality of storage locations 132, and plurality of cloud computing platforms 134. With reference thereto, the data source 201 may be configured to send, to the transform orchestrator 124, an order to transform biological data to one or more genomic biomarkers. Along with the order, the data source 201 may also be configured to send the biological data itself. Examples of the data source 201 may include the physician clinical records system 106, the pathology imaging system 108, the biological data repository 110, the radiomic imaging system 112, the provider genomic sequencers 116A, the partner genomic sequencers 1168, the organoid modeling labs 116C, the pathology lab/oncology system 118, and/or the primary care provider computer system 119.

The transform orchestrator 124 may include transform catalog 149, which may include transforms 150. As will be discussed elsewhere herein, a transform 150 may comprise a set of instructions that operate on an input file (e.g., a file including biological data) to determine an output file (e.g., a file including the genomic biomarker, disease state, or diagnostic result).

The transform orchestrator 124 may be configured to select, from the transform catalog 149, one or more transforms 150 that correspond to the order request. The transform orchestrator 124 associates each of the selected transforms 150 with cloud computing platforms 135 of the plurality of cloud computing platforms 134. This association may include, for example, providing a container image from the transform 150 to the associated cloud computing platform 135.

FIG. 2B illustrates an example implementation of the transform orchestrator 124, and further illustrates example processes of the transform orchestrator 124 intaking transforms 251 to build the transform catalog 149, and executing the transforms 251. With reference thereto, a transform database 250 may store transforms 251. It may be noted that although the example of FIG. 2B shows only one transform database 250, any number of transform databases may be used. It may further be noted that the transform database 250 may be part of the transform system 102; alternatively, the transform database 250 may be part of a system separate from the transform system 102.

In some implementations, the transform database 250 stores transforms 251 which are ready for execution when the correct configuration is available. For instance, validation may include having the transform 251 run against a variety of predetermined inputs; and, if each output matches an expected output, the transform 251 is validated. In other examples, validations may include validations that the transform: is based on an OS with proper support (e.g., supports an apt-get); supports certain commands (e.g., supports an “unshare” command); and does not already contain a particular directory. The transform database 250 may send transforms 251 to the transform catalog 149 for storage or to execution block 262 for execution.

The register/validation module 252 may also register and/or validate transforms to add them to the transform catalog 149. For example, an actor (e.g., a person, build system, etc.) may author a transform image 158 and related configuration. The actor may then use the register/validation module 252 to add the transform to the transform catalog 149. The register/validation module 252 may execute many validations to ensure that the transform is correctly formed. For example, as described above, validation may include having the transform 251 run against a variety of predetermined inputs; and, if each output matches an expected output, the transform 251 is validated. In other examples, validations may include validations that the transform: is based on an OS with proper support (e.g., supports an apt-get); supports certain commands (e.g., supports an “unshare” command); and does not already contain a particular directory.

Transforms 251 added to the transform catalog 149, and are thus accessible by a transform orchestrator 124. The transform catalog 149 may contain all transforms 251 that are known to the transform orchestrator 124. In some embodiments, a transform is included in the transform catalog 149 in order to be executed.

In some embodiments, the transforms 251 may be able to be added to, updated, and removed from the transform catalog 149 by a transform owner (e.g., the transform database 250). An update may occur in response to the transform owner making a material change in a computational process, because of a configuration update, or other modification. Any type of change may result in creating a new version of the transform 251, which may be used to update (overwrite) an existing entry in the transform catalog 149.

The transform orchestrator 124 may be configured such that once a transform 251 is added to the transform catalog 149, it will become active and available as a resource. Any triggers indicated in the transform system supplied configuration 154 will be set up, and will cause the transform catalog 149 to be executed.

In addition, in some embodiments, the transform image 158 may be a set of instructions used to create a container, wherein an example container 302 is shown in FIG. 3A. In some embodiments, the container 302 is a virtual compute environment where the instructions can be executed. In some embodiments, the transform image 158 is never executed, and does not have storage; whereas, the container 302 may be executed and may have storage.

In the illustrated example, the container 302 includes an input folder/directory 304 containing locations of where the inputs to the container are stored (e.g., a storage location 133). Container 302 further includes an executable 156. As will be explained in more detail elsewhere herein, the executable 156 may be an executable code portion of the transform 150, 251. The container 302 may also include an output folder/directory 308 where the outputs of the container are to be written. For example, the output folder/directory may be a storage location 133. In some embodiments, the input and output folders may be configured such that the transform orchestrator and/or containers do not need particular knowledge of where the folders are located. Such that the orchestrator can always populate and retrieve files from the input and output folders using a common addressing scheme based upon the transform itself (e.g. /transform_path/inputs and/transform_path/outputs). In other embodiments, where the underlying structure of the transform is not in a universal format, a harness may be configured for the transform such that a mapping of symbols is performed to present, for example, an expected uniformity (e.g. symbol/transform_path/inputs=address of input folder within container).

Moreover, some embodiments further include a docker layer to further facilitate intake for the containers 302. In an example, the docker layer and containers 302 may assist when a transform 150, 251 is received by the transform catalog 149, or sent from the transform catalog 149 to another component. However, this is only one example, and the docker layer and containers 302 may be used to assist sending a transform 150, 251 between any components.

In this regard, FIG. 3B illustrates an example of the docker layer 320, which may act as a platform for developing, shipping, and running the containers 302 of the transforms 150, 251. The docker layer 320 allows separation of the containers 302 from the necessary computing and data storage infrastructure so that the transforms 150, 251 may be delivered quickly upon receipt of an order to the transform orchestrator 124.

The container 302 may be a unit of software that packages code (e.g., the transform 150, 251 or other application), to thereby allow the packaged code to be deployed to and run on another computing environment. Furthermore, in some embodiments, there may be a container image, which may contain everything for running the transform 150, 251 (e.g., including code, runtime, system tools, system libraries and settings, etc.). In some implementations, the container image may become a container 302 at runtime.

The example of FIG. 3B further illustrates host operating system (OS) 325, which may be an OS running on a virtual machine (VM), e.g., a computer resource that uses software (rather than a physical computer) to run programs. Each VM may run its own OS, and function separately from other VMs (even when they are all running on the same host machine).

Returning now to the example of FIG. 2B, further illustrated are orchestrations 254, which are events or actions that cause a transform 251 to be executed by the transform orchestrator 124. These may be declared in the transform system supplied configuration 154 of the transform. The orchestration 254 may be triggered by manual trigger events 268 or by automatic events (e.g., a transform is automatically called from the transform catalog 149). An example manual trigger 268 may be a user selected HTTP call to transform orchestrator 124. An automatic trigger may, for example, be when a specific event 270 is transmitted from an workflow orchestrator 126 indicating that a particular order item is ready. An example implementation of a workflow orchestrator is described in U.S. patent application Ser. No. 16/927,976, titled “Adaptive Order Fulfillment and Tracking Methods and Systems”, and filed Jun. 13, 2020, which is incorporated herein by reference and in its entirety for all purposes. Additionally or alternatively, there may be another system event 272 that triggers the transform 251 to be executed. In some embodiments, the workflow orchestrator event 270 and other system event 272 are integrated at event integrations 274. In some embodiments, execution of the transform 251 is only triggered when a particular number or particular combination of events is reached at 274.

In some embodiments, the transform orchestrator 124 will only allow specifically configured triggers to begin a transform 251. For example, if a manual HTTP trigger is not configured for a given transform 251, calling that endpoint will not result in the transform 251 being executed.

A trigger's configuration may impact how input and outputs need to be handled.

In some embodiments, when it is determined that a transform 251 needs to execute, either through configured events or from a manual trigger, the transform orchestrator 124 will ensure the transform 251 is executed. The transform orchestrator 124 may manage the lifecycle and compute resources used to execute transforms 251.

In some implementations, when a transform 251 is triggered, the transform system 102 may add the transform 251 to a queue 256 (e.g., when the transform is triggered) and execute the transform 251 when computing resources become available. In some embodiments, the queue(s) 256 comprise more than one queue, such as a high priority queue and a low priority queue. In some examples, transforms 251 in the queue 256 may be executed and completed simultaneously. In some examples, transforms 251 may be executed and completed asynchronously.

The transform orchestrator 124 may apply computing resource-availability in determining and executing the ordering of the queue 256 (e.g., particular use of a high priority queue and a low priority queue), thereby allowing the transform system 102 to act robustly in the face of bursts of demand. For example, executions of transforms 251 may be treated as an asynchronous process by external callers even though a transform 251 is a synchronous process.

In some embodiments, the initial behavior of queue 256 may be based on a first in, first out (FIFO) management protocol against the available compute resources.

The transform orchestrator 124 may manage input and output files (e.g., through I/O management 258) on behalf of a transform 251. The I/O management 258 further advantageously may provide orchestration around input and output files. The configuration for a transform 251 may declare the inputs and outputs that are expected for a given execution.

Inputs and outputs may be data products. An input configuration may contain explicit files, or may be instructions/symbols on how to find files. The transform orchestrator 124 may reconcile the input declarations and may fetch them on behalf of the transform 251. The transform 251 itself may be able to simply use the files already present in the local file system, as they will be present when the computational process (e.g., at computation module 260) is started.

Each execution of a transform 251 is tracked (e.g., at monitoring 264). Each execution 262 run may have a unique identifier—an Execution Id—associated with it. This Execution Id is included in all events, notifications, logs and metrics that are associated with a given execution 262. This Execution Id provides the means to correlate events for the same execution.

In some embodiments, the transform 251 has different statuses (e.g., statuses of the execution 262), such as:
(i) In Progress The process has been started and is currently executing.
(ii) Complete The process has completed successfully.
(iii) Error The process has completed, but has not resulted in success. It may have crashed or otherwise indicated an error condition.
(iv) Submitted The process has been validated and created.
(v) Preparing-image The process is awaiting or undergoing the step to add the Transform Harness layer to the provided image.
(vi) Queued The process is ready to be run, and is waiting for compute resources to become available.
(vii) Starting The process has been submitted to an execution engine and is waiting to be started.
(viii) Fetching-input The process has started and is fetching data products, and/or other data that the Transform has configured.
(ix) Running The process is running.
(x) Writing output The process has executed successfully; and, for managed transforms, the Transform Harness is reading the output.json file and pushing any necessary data to the configured systems.
(xi) Succeeded The transform has completed successfully
(xii) Failed Any part of the Transform has failed to complete successfully. The error may be detailed in the error property of the Execution Record.

The transform orchestrator 124 makes these statuses, among others, available for a given execution of a transform 251, which may be done through notifications 266. For example, the transform orchestrator 124 may emit notifications at various stages of executing the transform 251. These notifications may be consumed by humans or machines to understand the state of a given execution. Example notifications may include, for example, the following data:
(i) Transform Name The name of the transform 251 being executed (e.g., 267A of the example of FIG. 2B). In one example, the transform name 267A is sent to the data source 201 when the transform begins to execute.
(ii) Execution ID The unique ID of the execution of a transform (e.g., 267B of the example of FIG. 2B). In one example, the Execution ID 267B is sent to the data source 201 when the transform begins to execute.
(iii) Status The status the transform execution 262 has most recently entered (e.g., 267C of the example of FIG. 2B). This minimally includes statuses In-Progress, Complete, and Error. More detailed statuses may be included if necessary, such as Queued, Fetching Inputs, and/or Writing Outputs. In one example, a status 267C, such as a final operational status, may be sent to the data source 201 when a determined genomic biomarker is stored in a storage location 133.
(iv) Timestamp The time when the Notification was emitted (e.g., 267D of the example of FIG. 2B). A RFC 3339 format timestamp with time zone and millisecond precision. In some examples, the timestamp 267D is simply sent along with other notifications.

Notifications may include additional data at various statuses. Examples of this may include error messages, listing of input files, listing of output files, etc.

In some embodiments, if a transform 251 is integrated with a workflow management system, such as the workflow orchestrator 126, the transform orchestrator 124 notifications, in some embodiments, are not used to determine next actions to take. In some embodiments transform orchestrator 124 notifications are used only for informational purposes, and workflow management signals from workflow orchestrator 126 are used for orchestration such as to determine next actions to take.

Returning to monitoring 264, in some embodiments, transform orchestrator 124 supports transform monitoring primarily through logging. When a transform 251 emits log statements, they may be published in a common location that is available to the transform owner (e.g., transform database 250). Log messages from transform orchestrator 124 regarding orchestration concerns (I/O management, status changes, success/failure modes) may be available in the same location. In some embodiments, all log messages for a single execution may be correlated by the Execution ID discussed elsewhere herein.

In addition to monitoring the transform 251, the transform orchestrator 124 (e.g., at monitoring 264 or anywhere else) may monitor itself. For example, transform orchestrator 124 may publish events as logs and metrics during various stages of its execution. That logging may help drive visibility to questions like number of running transforms, number of transforms that errored out within a certain time range, etc.

The transform orchestrator 124 also performs error handling (e.g., through error handling module 265 of the monitoring module 264). In some embodiments, there are different types of errors, such as validation errors, orchestration errors, execution errors, and/or timeout errors.

Regarding validation errors, the transform orchestrator 124 may validate a transform 251 when it is registered into the transform catalog 149. If the transform system supplied configuration 154 is not valid, an error may be returned to the user indicating the problem. The same error checking may be done when a transform 251 is updated.

In some embodiments, tooling may be available to help a team ensure their packaged transform 251 will behave as expected. This includes validations that the configuration is correct and that necessary components are present.

In some implementations, the tooling may also support the engineers when developing test cases for their transform 251. A useful feature may be to run the transform 251 against a variety of predetermined inputs, and to validate that the output matches an expectation in each case. This may be done as part of the registration process when the transform 251 is registered to the transform catalog 149.

Regarding orchestration errors, the transform system 102 may provide a number of orchestration features including retrieval of input files 332, storage of output files 334, trigger management, notifications, and running the transforms 251. The transform system 102 may attempt to retry any of these functions if they fail, when appropriate. If the function ultimately fails, a log statement and notification may be emitted outlining the exact problem that occurred. Some orchestration errors may additionally be reflected in integrated workflow management systems, such as workflow orchestrator 126.

Regarding execution errors, execution of a transform 251 may result in a failure or crash. Each of these conditions will be detected by transform orchestrator 124 and may be logged and emitted as a notification. These errors may be reflected in integrated workflow management systems when applicable, such as workflow orchestrator 126.

Regarding timeout errors, execution of a transform 251 may result in the computational process hanging indefinitely. The transform system supplied configuration 154 may require defining a maximum execution time. If this maximum amount of time has elapsed since the process was started, it may be aborted and a log message and notification may be emitted. This error may be reflected in integrated workflow management systems, such as workflow orchestrator 126.

### Example Transforms

An example implementation of a transform and how it is triggered is provided below. In various embodiments, transform is a computational process designed to receive well-defined input files and perform some computation based on only the received input files, without dependencies on external databases, APIs, or other external infrastructure. In some examples, reference data sets may be specified as input files utilized by the computation process. In either example, the transform produces new information as specific, predictable, well-defined output files.

In this regard, the transform may be an intentionally constrained solution space. If additional infrastructure may be needed to facilitate execution of the transform, that infrastructure may be determined by the transform orchestrator 124 as described in examples herein.

Thus, in some examples, a transform is configured based on two principles: dependency-free execution and declarative input and output files. Declaring well-defined inputs and outputs allows the transform system to manage fetching and storage of input and output files, while removing knowledge of network file storage mechanisms. Executing without utilizing dependencies, such as databases, APIs, etc., allows the transform system to have unlimited horizontal scalability while minimizing external bottlenecks. Eliminating dependencies also allows for more efficient testing of transforms in isolation from other transforms, which can provide regulatory benefits as well.

In some example implementations, one or both of the author supplied configuration 155 and/or the transform system supplied configuration 154 may be a JavaScript Object Notation (JSON) file. In this regard, it should be understood that a JSON file format is a language-independent data format. Further, the JSON file format may be a data interchange format using human-readable text to store and transmit data objects including attribute—value pairs and arrays or other serializable values.

FIG. 3C illustrates an example application of a transform 350 (e.g., which may be executed by the transform orchestrator 124). In some embodiments, the transform 350 takes input files 332 as inputs, which may comprise the biological data, such as received from a data source 201 and/or the database 140.

The input files 332 may comprise any suitable file format. For instance, if the input files contain nucleic acid reads, the file format may be a FASTQ format (e.g., a text-based format for storing both a biological sequence, such as nucleotide sequence, and its corresponding quality scores) or a BAM format. In another example, if the input files contain a slide image, the file format may be any image file format such as Joint Photographic Experts Group (JPEG), Tagged Image File Format (TIFF), Windows bitmap (BMP), Portable Network Graphics (PNG), etc. Moreover, any combination of file formats may be used in the input files 332. For example, if the input files 332 comprise both nucleic acid reads and a slide image, the input files 332 may contain a first file format (e.g., FASTQ or BAM) for the nucleic acid reads, as well as a second file format for the slide image (e.g., an image file format). In some examples (e.g., relating to a cardiac diagnosis), the input files may comprise cardio trace data, such as from an electrocardiogram (ECG), and such as in the form of a comma-separated values (CSV) file.

The transform 350 may output the output files 334, which may comprise the determined genomic biomarker. In some embodiments, the output files 334 are written to a storage location 133 of the plurality of storage locations 132. The output files 334 may be in any suitable format to indicate the genomic biomarker. The file formats may also vastly differ between each transform 350. In some examples, the output files 334 have the same file format as the input files 332. Examples of the file formats of the output files 334 include: Variant Call File (VCF), FastQ, Binary Aligned Map (BAM), tabular/comma separated (TSV/CSV), and JSON. In some embodiments, these file formats may be described in a Data Product specification (a Specification), and encapsulated into a Data Product data structure. The Specification may document the file's shape, format, and syntax, and have a Policy that defines which users and groups can create, read, or query the file.

In some embodiments, the Specifications are stored in a Data Products Service (DPS). When a transform has completed, it has files in its output folder, and a list of Data Product Types that match to those files. The service checks that the files in fact validate against the Specification as stored in the DPS and then provides a cloud URL to which that file is uploaded. That URL is indexed within the DPS, and if someone wants to retrieve it, the DPS is used to search for and manage access to it. There may also be an output.json file written by the transform while executing which maintains a mapping of files to types.

In some examples, the output files 334 include file type(s) that is/are aligned with a Specification that documents the file's shape, format, and syntax, and has a Policy defining which users and groups can create, read, or query the file. Specifications may be stored in a Data Product Service (DPS). When a transform 350 has completed, it has files in its output folder, and a list of Data Product Types that match to those files. The service may check that the files in fact validate against the Specifications as stored in the DPS and then provide a cloud URL to which that file is uploaded. That URL may then be indexed within the DPS; and, if an entity wants to retrieve it, the DPS may be used to search for and manage access to it. In one example, an output.json file may be written by the transform 350 while executing which maintains a mapping of files to types.

Some implementations also include transform system supplied configuration 354 and executable 356. In some embodiments, the transform system supplied configuration 354 may comprise a set of instructions for how the transform behaves during execution. The transform system supplied configuration 354 may cover areas including:
Transform registration/metadata, including unique name, owner, etc.
Information necessary to identify and fetch inputs
Information necessary to permanently store outputs
Definitions of trigger(s) which should cause the transform 350 to begin
Instructions to execute the transform's executable 356

Some examples of information that the transform system supplied configuration 354 may include are: (i) a unique ID for the transform 350 (which is stored in the transform catalog); (ii) a specification of a cloud computing platform 135 (and related configuration required by the cloud computing platform 135) that will be used for a specific execution of the transform 350; (iii) parameters (pointer, authorization credentials, etc.) for the transform's communication interface 352, so that it is able to communicate with the transform system 102 while the transform 350 is running in the plurality of cloud computing platforms 134; (iv) authorization credentials needed to access other APIs within the transform system 102 or within the plurality of cloud computing platforms 134; and/or (v) details about how or where to send log files or metrics.

In some embodiments, separate from the transform system supplied configuration 354, there may be a transform author supplied configuration 355, which may include details of the transform 350. Some examples of information that the author supplied configuration 155 may include are: (i) a unique name of the transform; (ii) an indication of the author or owner; (iii) a specification of the author's executable/container image containing their executable; (iv) specification of the input and output parameters; (v) input and output parameters that may indicate persistent volumes/storage locations; (vi) specification of the amount and type of compute resources required to run the executable (CPU, memory, disk space); and/or (vii) specification of signals from the workflow orchestrator 126 which: (a) cause the transform to be executed, and (b) optionally include instructions on how to identify input parameters from data stored within the workflow orchestrator 126.

Unless specified otherwise, “configuration” may refer to either the transform system supplied configuration or the author supplied configuration.

The transform 350 may also include the executable 356, which may be the computational process that embodies the functionality of the transform 350 (e.g., the executable code). The computational process may be implemented using the language and frameworks of a development team's choosing.

In some embodiments, the executable 356 may have knowledge of or access to a symbol identifying one directory where its input files 332 will be available. In some embodiments, the executable 356 may also have knowledge of or access to a symbol identifying one directory where its output files 334 may be written. Additionally, in some embodiments, the input and output directories may be provided at runtime by the transform system 102.

The executable 356 may be the computational mechanism that converts the input files 332 to the output files 334. The process may be implemented in the language or framework of the engineering team's choosing. In some embodiments, it may have all code dependencies pre-fetched or compiled so that its behavior cannot change on subsequent executions. The executable 356 of the transform 350 may be executed by the transform orchestration 124.

In some embodiments, the executable 356 may have knowledge of one directory where its input files 332 will be available. In some embodiments, it may also have knowledge of one directory where its output files 334 may be stored. Details surrounding these directories will be discussed elsewhere herein. A transform orchestrator may ensure that configured input files 332 are placed in the input directory, and output files 334 are captured from the output directory and are written to permanent storage (e.g., a storage location 133 of the plurality of storage locations 132).

In some implementations, the executable 356 may provide monitoring output via a logging mechanism (e.g., monitoring 264). In some implementations, the monitoring may be supported by the transform system supplied configuration 354, container 302 and/or other infrastructure within the cloud computing platform 135 in order to make output such as logging available to interested parties.

In some embodiments, the executable 356 may provide a reliable communication of success or error conditions that may occur during execution. The transform orchestrator may use this information to facilitate communications with the team and systems involved in the Business Process.

In some embodiments, transform system supplied configuration 354 may ensure that configured input files 332 are placed in the input directory, and output files 334 are captured from the output directory and are written to permanent storage (e.g., in memory 122).

In some implementations, the executable 356 may provide logging output via a standard output (stdout) 338 and standard error (stderror) 340. It should be understood that stdout 338, e.g., standard output, may be a default descriptor where the executable 356 may write certain outputs, such as logging outputs. It should be further understood that stderror 340 may be may be a default descriptor where the executable 356 may write certain outputs, such as error logging outputs.

In some embodiments, the executable 356 may provide a reliable communication of success or error condition by returning an exit code 336. Some embodiments support the following exit codes: (i) 0 for success, and (ii) any positive number for failure. In some embodiments, the exit code 336 is provided to a data source 201 to inform the data source 201 of the success or failure.

In some embodiments, the transforms 350 may be executed in a programming language agnostic way. In order to abstract the programming language away, a docker container, such as container 302 from the examples of FIGS. 3A and 3B, may be used to encapsulate the executable code. In some embodiments, the executable code includes the executable 356, as well as platform-provided orchestration code.

Furthermore, in some embodiments, the transform 350 includes communication interface 352, and/or transform image 358.

Figures and embodiments disclosed herein are exemplary in nature for depicting various implementations supported within the current disclosure and may be combined in part or whole. For example, components from any of the figures may be combined with components from other figures.

### Example Methods

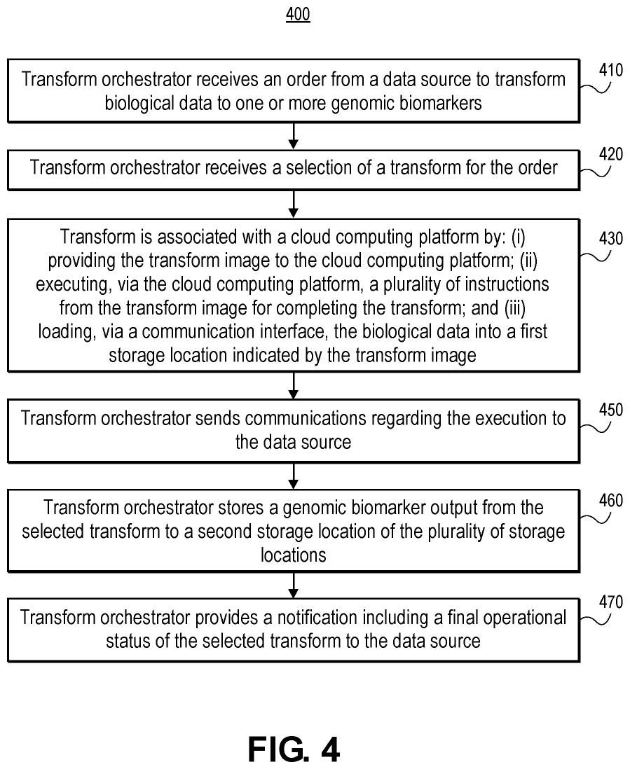

FIG. 4 is a flowchart of an example method of transforming biological data to one or more genomic biomarkers as may be achieved using the precision medicine architecture 100. The example method 400 begins at block 410 when the transform orchestrator 124 receives an order from a data source 201 to transform biological data to one or more genomic biomarkers. Examples of the biological data include nucleic acid reads (e.g., reads of DNA or RNA), and/or images (e.g., slide images, H&E stain images, IHC images, radiology images, etc.). However, it should be understood that any biological data may be used.

In implementations where the biological data comprises nucleic acid reads, the nucleic acid reads may be determined by any suitable method. For example, the nucleic acid reads may be determined by next generation sequencing, which, as is understood in the art, may be sequencing technology with high throughput, scalability, and speed. The nucleic acid reads may be in any suitable format (e.g., a FASTQ format or a BAM format, etc.).

In some embodiments, the biological data comprises a specific number of, or range of nucleic acid reads (e.g., 10,000 or more nucleic acid reads). Furthermore, the nucleic acid reads may come from any suitable specimen (e.g., any specimen from a human, animal, plant, virus, or bacteria). The nucleic acid reads may be aligned or unaligned to a common reference genome.

In addition, in some embodiments, the order may be received along with the biological data itself. For example, the data source 201 may send the order and the biological data together.

Examples of the genomic biomarker(s) to be determined include: a microsatellite instability, a tumor mutational burden, a variant characterization, a copy number variation, a fusion, or a presence of a pathology/tissue stain image-derived biomarker (e.g., an IHC slide stained image, H&E slide stained image, etc.). However, it should be understood that any genomic biomarker may be determined.

At block 420, the transform orchestrator 124 receives a selection of a transform 150 (e.g., from a data source 201, or any other suitable component) for the order.

The selection of the transform 150 for each genomic biomarker may be made based on any suitable criteria. For example, the transform 150 may be explicitly selected by a person or system responsible for ensuring that the genomic biomarker is being produced. In another example, a specific transform 150 may be triggered (thus “selecting” the transform 150) by a signal from the workflow orchestrator 126, based on the author specified configuration 155.

In another example, the selection of the transform 150 may be made based on compute requirements of the order to transform the biological data. For instance, the selection of the transforms may be made based on an available VM memory size, central processing unit (CPU) performance, graphics processing unit (GPU) performance, and/or a resource quota. An example of the resource quota includes a constraint on total compute resources available to a group of transforms. In some implementations, when a particular resource quota is reached, additional incoming orders will be sent to the queue 256.

At block 430, the transform orchestrator 124 associates each selected transform 150 with a cloud computing platform 135. It should be understood that any cloud computing platform 135 of the plurality of cloud computing platforms 134 may be implemented on a single processor, or across multiple processors. In some embodiments, a platform is implemented as a virtual machine VM.

The association may be made based on any suitable criteria. For example, the association may be made based on the transform system supplied configuration 154. For example, the transform system supplied configuration 154 may specify a particular cloud computing platform 135, or criteria of a platform (e.g., resource requirements of the transform, etc.). The association increases the transform system 102's ability to uniformly process each transform (e.g., every transform will be able to be processed), which is an advantage over the prior art.

The associating at block 430 may further include: providing the transform image 158 to the associated cloud computing platform 135; and executing the instructions of the transform image 158 at the cloud computing platform 135.

The associating at block 430 may further include loading (e.g., via the communication interface 125 and/or the communication interface 152) the biological data into a first storage location of the plurality of storage locations 132. Further at block 430, the transform orchestrator 124 may provide the transform image 158 to the cloud computing platform 135.

In some embodiments, further at block 430, the transform orchestrator 124 may determine if an execution error has occurred (e.g., as described elsewhere herein). If an execution error has occurred, the transform orchestrator 124 may send a notification to the data source 201, or any other suitable component.

At block 450, the transform orchestrator 124 may send communications regarding the execution to the data source 201, or any other suitable component. The communications may include the operational statuses of each selected transform. Examples of the operational status include:
(i) In Progress The process has been started and is currently executing.
(ii) Complete The process has completed successfully.
(iii) Error The process has completed, but has not resulted in success. It may have crashed or otherwise indicated an error condition.

At block 460, the transform orchestrator 124 may store a genomic biomarker output from each selected transform to a second storage location of the plurality of storage locations 132. Additionally or alternatively, the output genomic biomarker may be stored in an external database, such as database 140.

At block 470, the transform orchestrator 124 may provide a notification including a final operational status of each selected transform to the requesting data source 201, or any other suitable component. In some embodiments, the final operational status may be any of the operational statuses described elsewhere herein. In some implementations, the provision of the final operational status is based on the storing of the genomic biomarker. For example, once the output genomic biomarker is successfully stored, a final operational status of complete is provided to the data source 201.

Furthermore, the notification may include a notification of an error as described elsewhere herein. For example, the notification may include notification of an orchestration error, an execution error, or a timeout error, etc.

Moreover, at any point throughout the example method 400, the transform orchestrator 124 may provide a log statement to the data source 201. The log statement may include: (i) a currently running number of transforms, and/or (ii) a number of transforms flagged with errors during a particular time period.

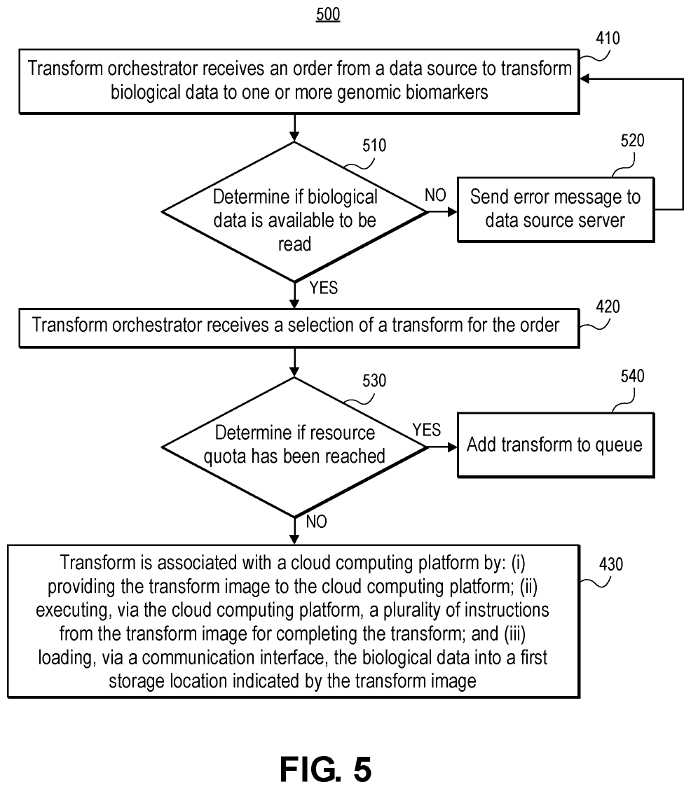

FIG. 5 is a flowchart of an example method 500 relating to the transform orchestrator 124 receiving an order and biological data from a data source server 201. With reference thereto, the example method begins at block 410, which may be performed similarly to block 410 of the example of FIG. 4.

At decision block 510, the workflow orchestrator 126, or the transform orchestrator 124 may determine if the biological data corresponding to the order is available to be read. In some embodiments, the workflow orchestrator 126 hosts a “ready state” for the biological data, thereby facilitating determination of if the biological data is available to be read. In some implementations, the determination is made by the workflow orchestrator 126, and/or the transform orchestrator 124 listening for necessary inputs to become available, and then sending a signal to execute when the inputs are available. Additionally or alternatively, an external orchestrator (e.g., outside of the transform system 102) may determine that all necessary inputs are available, and send the signal to the transform system 102 to execute.

If the biological data is not available to be read, the workflow orchestrator 126, or transform orchestrator 124 may send an error message to any of the data source 201 at block 520. The error message may include any relevant information. For example, the error message may include the order id and information regarding why the biological data is not available to be read. The method 500 may then return to block 410 where the data source 201 may send a new order and optionally send biological data. Alternatively, the data source 201 may resend the previous order along with new biological data in an attempt to fix the error.

If the biological data is available to be read at decision block 510, the transform orchestrator 124 performs block 420 similarly to block 420 of FIG. 4 (e.g., selects a transform for each of the received genomic biomarkers).

At decision block 530, the transform orchestrator 124 determines if an overall resource quota has been reached (e.g., a memory, CPU, and/or GPU quota). If so, at block 540, the transform orchestrator 124 may add the transform 150 to the queue 256. In some embodiments, at block 540, the transform orchestrator 124 further determines if the transform 150 should be added to a high priority queue or a low priority queue, and adds the transform 150 to the appropriate queue.

If, at decision block 530, it is determined that the resource quota has not been reached, the method proceeds to block 430, which may be performed similarly to block 430 of FIG. 4. From there, the example method 500 may proceed according to the example method 400 of FIG. 4 (e.g., by performing blocks 450, 460, and 470 of FIG. 4).

Additionally or alternatively to block 530, in variations where multiple queues are used, there may be resource quotas associated with each queue; and, in some further variations, transforms 150 may be added to the queues, but then dequeued based on resource availability.

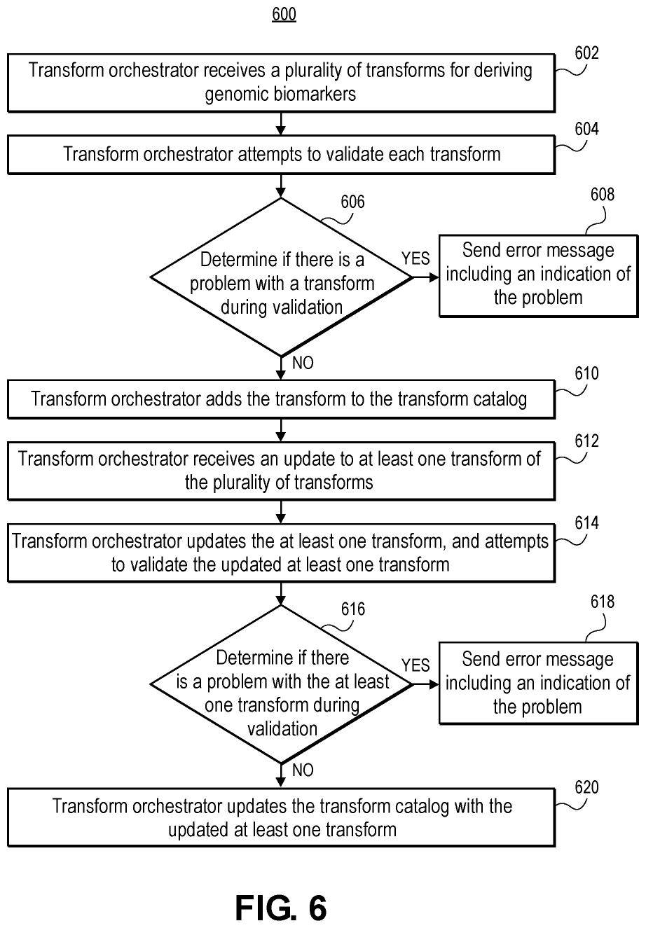

FIG. 6 illustrates an example method 600 for creating a transform catalog 149. With reference thereto, at block 602, the transform orchestrator 124 receives a plurality of transforms 150 (e.g., from the transform database 250). At block 604, the transform orchestrator 124 may attempt to validate each transform 150.

At decision block 606, the transform orchestrator 124 may determine if there is a problem with a transform 150 during validation. If so, at block 608, an error message (e.g., a validation error message) may be sent to the transform database 250, or any other suitable component. In embodiments where the transform database 250 is part of the transform system 102, an indication of the error may be displayed on user interface 123.

If, at decision block 606, it is determined that there is no problem with the transform, the transform orchestrator 124 adds the transform 150 to the transform catalog 149 at block 610.

At block 612, the transform orchestrator 124 receives an update to at least one transform of the plurality of transforms. At block 614, the transform orchestrator 124 updates the at least one transform, and attempts to validate the updated at least one transform.

At decision block 616, the transform orchestrator 124 determines if there is a problem with the updated at least one transform during validation. If so, at block 618, an error message (e.g., a validation error message) may be sent to the transform database 250, or any other suitable component. In embodiments where the transform database 250 is part of the transform system 102, an indication of the error may be displayed on user interface 123.

If, at decision block 616, it is determined that there is no problem with the transform 150, the transform orchestrator 124 adds the updated at least one transform to the transform catalog 149 at block 620.

Further regarding the example flowcharts provided above, it should be noted that all blocks are not necessarily required to be performed. Moreover, additional blocks may be performed although they are not specifically illustrated in the example flowcharts. In addition, block(s) from one example flowchart may be performed in another of the example flowcharts.

Additional Example Transform System

FIG. 7 illustrates an example system 700 and transform system 702 that may be used to implement the architecture 100 and transform system 102 of FIG. 1. It should be understood that the components of FIGS. 1 and 7 may be implemented additionally or alternatively to each other. For instance, any transform system 102, 702 may have some components from both figures, and further may not require all components from either figure.

As illustrated in the example of FIG. 7, the transform system 702 may include one or more processing units 710, which may represent Central Processing Units (CPUs), and/or on one or more or Graphical Processing Units (GPUs) 711, including clusters of CPUs and/or GPUs, and/or one or more tensor processing unites (TPU) (also labeled 711), any of which may be cloud based. Features and functions described for the transform system 702 may be stored on and implemented from one or more non-transitory computer-readable media 712 of the transform system 702. The computer-readable media 712 may include, for example, an operating system 714, transform orchestrator 724, and workflow orchestrator 726. More generally, the computer-readable media 712 may use the components it includes to implement the techniques described herein. The computer-readable media 712 and the processing units 710 and TPU(S)/GPU(S) 711 may store the biological data, the genomic biomarkers, or any other data in one or more databases 713.

The transform system 702 includes a network interface 750 communicatively coupled to the network 704, for communicating to and/or from a portable personal computer, smart phone, electronic document, tablet, and/or desktop personal computer, or other transform systems. The transform system 702 further includes an I/O interface 752 connected to devices, such as digital displays 728, user input devices 730, etc. In some examples, as described herein, the transform system 702 generates biomarker prediction as an electronic document 715 (such as the output files 334) that can be accessed and/or shared on the network 704. In the illustrated example, the transform system 702 is implemented as a single transform system 702. However, the functions of the transform system 702 may be implemented across distributed devices 702, 704, etc. connected to one another through a communication link or through the network 704. In other examples, functionality of the transform system 702 may be distributed across any number of devices, including the portable personal computer, smart phone, electronic document, tablet, and desktop personal computer devices shown. In other examples, the functions of the transform system 702 may be cloud based, such as, for example one or more connected cloud TPU(s) customized to perform machine learning processes. The network 704 may be a public network such as the Internet, private network such as a research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network 704 can include a number of devices that facilitate network communications and/or form a hardware basis for the networks, such as switches, routers, gateways, access points (such as a wireless access point as shown), firewalls, base stations, repeaters, backbone devices, etc.

The computer-readable media 712 may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the transform system 102 may represent a CPU-type processing unit, a GPU-type processing unit, a TPU-type processing unit, a field-programmable gate array (FPGA), another class of digital signal processor (DSP), or other hardware logic components that can be driven by a CPU.

Thus, as provided, a system 700 for performing the methods described herein may include a transform system 702, and more particularly may be implemented on one or more processing units, for example, Central Processing Units (CPUs), and/or on one or more or Graphical Processing Units (GPUs), including clusters of CPUs and/or GPUs. Features and functions described may be stored on and implemented from one or more non-transitory computer-readable media 712 of the transform system 702. The computer-readable media 712 may include, for example, an operating system 714 and software modules, or “engines,” that implement the methods described herein. More generally, the computer-readable 712 media may store batch normalization process instructions for the engines for implementing the techniques herein. The transform system may be a distributed computing system, such as an Amazon Web Services cloud computing solution.

Plurality of cloud computing platforms 731 may also be connected to the network 704, and may include cloud computing platforms 735. The cloud computing platforms 735 may include a plurality of storage locations 732 including individual storage locations 733. It should be understood that a cloud computing platforms 735 may be any hardware, software, or combination thereof used to host an application or service. In one example, a platform includes hardware, an OS, and coordinating programs using an instruction set for a specific microprocessor. In some embodiments, a platform may have different abstraction levels, such as a computer architecture, an OS, and/or a runtime library. It should further be understood that certain algorithms may be platform-specific (e.g., it was developed to run on a certain platform), cross-platform, or platform-agnostic.

The functions of the engines may be implemented across distributed transform systems, etc. connected to one another through a communication link. In other examples, functionality of the system may be distributed across any number of devices, including the portable personal computer, smart phone, electronic document, tablet, and desktop personal computer devices shown. The transform system may be communicatively coupled to the network and another network. The networks may be public networks such as the Internet, a private network such as that of a research institution or a corporation, or any combination thereof. Networks can include, local area network (LAN), wide area network (WAN), cellular, satellite, or other network infrastructure, whether wireless or wired. The networks can utilize communications protocols, including packet-based and/or datagram-based protocols such as Internet protocol (IP), transmission control protocol (TCP), user datagram protocol (UDP), or other types of protocols. Moreover, the networks can include a number of devices that facilitate network communications and/or form a hardware basis for the networks, such as switches, routers, gateways, access points (such as a wireless access point as shown), firewalls, base stations, repeaters, backbone devices, etc.

Additional Example Transform Signal Diagram

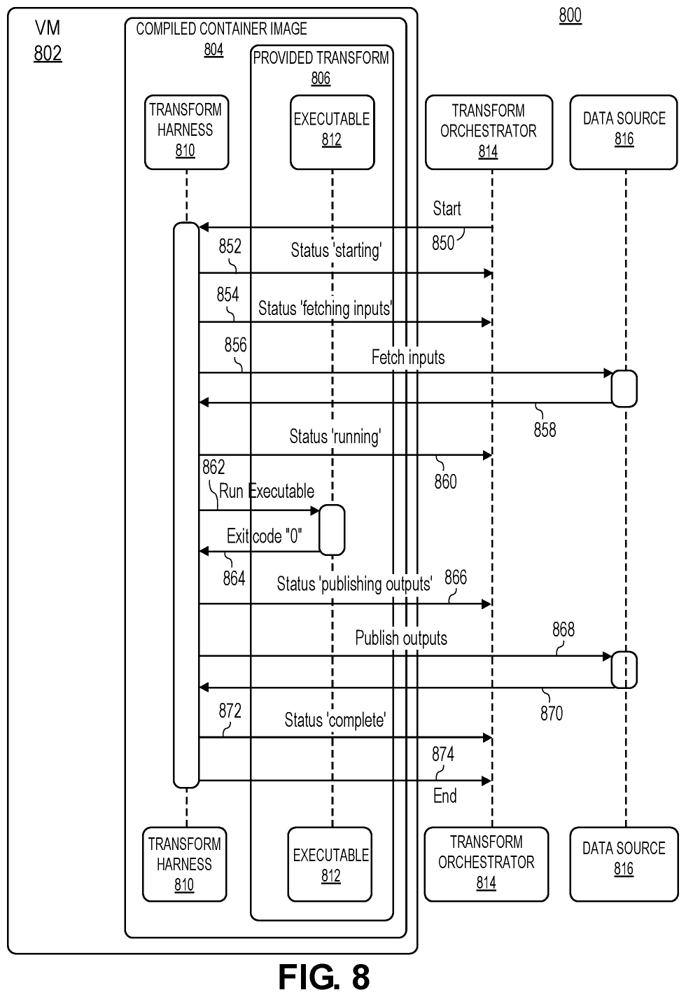

FIG. 8 illustrates an example signal diagram 800 for applying a transform 806. It should be understood that the events and components of FIG. 8 may be implemented additionally or alternatively to events and/or components of any other figure.

The example signal diagram 800 begins at event 850 when the transform orchestrator (e.g., running on VM 802) 814 sends a start command to the transform harness 810 (e.g., which may be part of the compiled container image 804 running on the VM 802, and which may be a configuration, such as the transform system supplied configuration 354). In one example, the transform harness 810 is or comprises a docker layer, such as the docker layer 320. In some implementations, the start command comprises an order to transform biological data to a biomarker (e.g., a genomic biomarker). Along with the start command, the transform orchestrator 814 may also send the biological data itself.

At event 852, the transform harness 810 sends a status (e.g., operational status) of “starting” to the transform orchestrator 814. In some implementations, the transform harness 810 then checks the transform 806 for errors as it is starting up. Such startup errors may include erroneous requests, including requester authentication, bad input parameters, etc.; and these errors may be handled before the transform 806 is triggered. These errors may also be indicated directly to the requester. Additionally or alternatively, the startup errors may be errors attempting to set up the transform communication interface, or errors occurring when using the communication interface to call APIs to initialize shared state between the executing transform 806 (within the cloud platform 135, 735) and the Transform Service. These may be due to faulty transform system provided configuration, network outages, etc.

If there are no errors upon startup (e.g., the startup is successful), then the transform harness 810 sets the status to “fetching inputs” at event 854).

The transform harness 810 may then fetch the inputs (e.g., input files 332) from the data source 816 at events 856, and 858. Once the inputs have been fetched, the transform harness 810 may set the status to “running” at event 860.

The transform harness 810 may then run the executable 812 at event 862. In the example of FIG. 8, the executable 812 runs successfully, and thus produces an exit code of “0” (which indicates success, similarly to the example exit code 336 of FIG. 3C) at event 864.

The transform harness 810 may then set the status to “publishing outputs” at event 866, and may then publish the outputs at event 868. To publish the outputs, the transform harness 810 may send the output files, such as the output files 334 of FIG. 3C, to the data source 816. At event 870, the data source 816 indicates to the transform harness 810 if the publication was successful at event 870.

At event 872, the transform harness 810 may then set the status to “complete.” The transform harness may then indicate to the transform orchestrator 814 that the process has ended at event 874.

Transform Examples

To further illustrate, the following will describe non-limiting examples of transforms.

In one example the transform is a slide import pipeline transform. This transform may import and process newly scanned slides (e.g., slide images, H&E stain images, IHC images) so they can be available in a digital-path viewer. This transform may take SVS (ScanScope Virtual Slide) or TIFF (Tag Image File Format) files as inputs. This transform may output a deepzoom pyramid of a slide, a macro image of the slide, label image of the slide, or a metadata.json extracted from original image.

In another example, the transform is a slide scrubbing transform. This transform removes label image, macro image, and metadata containing info considered PHI (protected health information) from scanned slide images. Once images pass this process they are able to be delivered to partners or used for data-science. This transform may take SVS or TIFF files as inputs. The outputs may also be SVS or TIFF files.

In another example, the transform is a slide classification transform. This transform runs data-science models to classify tissue and perform cell segmentation, and also performs marker detection and tissue detection for QC (quality control) processes. This transform may take SVS files, TIFF files, or files indicating a cancer type as inputs. Examples of output files for this transform include files indicating issue detection results, slide QC marker results, slide QC tissue masks, and slide QC marker run statuses.

In another example, the transform is a slide pyramid transform. This transform generates image pyramid masks depicting tissue classification and cell segmentation results. These pyramids may be used to visually overlay the results on top of the original slide in the pathology viewer. This transform may take raw output files from slide classification runs as inputs. An example of an output of this transform is an overlay pyramid.

In another example, the transform is a slide QC transform. This transform runs data science models to analyze if a slide has detectable blurriness or tissue folds as part of an automated QC process. Examples of inputs to this transform include a TIFF file, a SVS file, a slide tissue mask, and a slide marker mask. Examples of outputs for this transform include data products, such as slide QC clustering blur results, slide QC laplacian blur results, slide QC clustering folds results, slide QC clustering blur masks, slide QC clustering folds masks, slide QC clustering blur run statuses, slide QC laplacian blur run statuses, and slide QC clustering folds run statuses.

In another example, the transform is a fusion align transform. As an input, this transform may take a URL link to a FASTQ file. This transform may generate a BAM file having aligned RNA transcripts. Additionally or alternatively, this transform may generate an output comprising identified chimerics, splices, and fusions.

In another example, the transform is a QC transform. As an input, this transform may take a URL link to a FASTQ file. This transform may generate FASTQ files listing trimmed forward and reverse nucleotide chains.

In other examples, the transform is a fusion star transform or a mojo transform. These transform may use an input FASTQ file to generate a fusion according, respectively, to either a fusion star method or a mojo method. The generated fusion may, in some examples, be used as an input to an annotation transform. The annotation transform may use this input to generate an annotated fusion with breakpoint location and domain of protein.

In another example, the transform is a RNA expression alignment transform. This transform may take inputs of FASTQ files, and output spliced junctions, BAM files having file aligned RNA transcripts, and QC summary statistics.

In another example, the transform is an IHC prediction transform. This transform may take an input of an SRE (solid RNA expression) file and output an IHC prediction, for example, in CSV (comma separated values) format.

Additional Information

Some embodiments may include a genetic analyzer system, and the genetic analyzer system may include targeted panels and/or sequencing probes. An example of a targeted panel is disclosed, for example, in U.S. Patent Publication No. 2021/0090694, titled “Data Based Cancer Research and Treatment Systems and Methods”, and published Mar. 25, 2021, which is incorporated herein by reference and in its entirety for all purposes. An example of a targeted panel for sequencing cell-free (cf) DNA and determining various characteristics of a specimen based on the sequencing is disclosed, for example, in U.S. patent application Ser. No. 17/179,086, titled “Methods And Systems For Dynamic Variant Thresholding In A Liquid Biopsy Assay”, and filed Feb. 18, 1921, U.S. patent application Ser. No. 17/179,267, titled “Estimation Of Circulating Tumor Fraction Using Off-Target Reads Of Targeted-Panel Sequencing”, and filed Feb. 18, 1921, and U.S. patent application Ser. No. 17/179,279, titled “Methods And Systems For Refining Copy Number Variation In A Liquid Biopsy Assay”, and filed Feb. 18, 1921, which are incorporated herein by reference and in their entirety for all purposes. In one example, targeted panels may enable the delivery of next generation sequencing results (including sequencing of DNA and/or RNA from solid or cell-free specimens) according to an embodiment, above. An example of the design of next-generation sequencing probes is disclosed, for example, in U.S. Patent Publication No. 2021/0115511, titled “Systems and Methods for Next Generation Sequencing Uniform Probe Design”, and published Jun. 22, 2021 and U.S. patent application Ser. No. 17/323,986, titled “Systems and Methods for Next Generation Sequencing Uniform Probe Design”, and filed May 18, 1921, which are incorporated herein by reference and in their entirety for all purposes.

Some embodiments may include an epigenetic analyzer system, and the epigenetic analyzer system may analyze specimens to determine their epigenetic characteristics and may further use that information for monitoring a patient over time. An example of an epigenetic analyzer system is disclosed, for example, in U.S. patent application Ser. No. 17/352,231, titled “Molecular Response And Progression Detection From Circulating Cell Free DNA”, and filed Jun. 18, 1921, which is incorporated herein by reference and in its entirety for all purposes.

Some embodiments may include a bioinformatics pipeline, and the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting DNA and/or RNA read counts aligned to a reference genome.

Some embodiments may include a RNA data normalizer (or any other molecular data normalizer), and any RNA read counts may be normalized before processing embodiments as described above. An example of an RNA data normalizer is disclosed, for example, in U.S. Patent Publication No. 2020/0098448, titled “Methods of Normalizing and Correcting RNA Expression Data”, and published Mar. 26, 2020, which is incorporated herein by reference and in its entirety for all purposes.

Some embodiments may include a genetic data deconvolver, and any system and method for deconvolving may be utilized for analyzing genetic data associated with a specimen having two or more biological components to determine the contribution of each component to the genetic data and/or determine what genetic data would be associated with any component of the specimen if it were purified. An example of a genetic data deconvolver is disclosed, for example, in U.S. Patent Publication No. 2020/0210852, published Jul. 2, 2020, and PCT/US19/69161, filed Dec. 31, 2019, both titled “Transcriptome Deconvolution of Metastatic Tissue Samples”; and U.S. patent application Ser. No. 17/074,984, titled “Calculating Cell-type RNA Profiles for Diagnosis and Treatment”, and filed Oct. 20, 2020, the contents of each of which are incorporated herein by reference and in their entirety for all purposes.

RNA expression levels (or other molecular data levels) may be adjusted to be expressed as a value relative to a reference expression level. Furthermore, multiple RNA expression data sets may be adjusted, prepared, and/or combined for analysis and may be adjusted to avoid artifacts caused when the data sets have differences because they have not been generated by using the same methods, equipment, and/or reagents. An example of RNA data set adjustment, preparation, and/or combination is disclosed, for example, in U.S. patent application Ser. No. 17/405,025, titled “Systems and Methods for Homogenization of Disparate Datasets”, and filed Aug. 18, 2021.

Some embodiments may include an automated RNA expression caller, RNA expression levels associated with multiple samples may be compared to determine whether an artifact is causing anomalies in the data. An example of an automated RNA expression caller is disclosed, for example, in U.S. Pat. No. 11,043,283, titled “Systems and Methods for Automating RNA Expression Calls in a Cancer Prediction Pipeline”, and issued Jun. 22, 2021, which is incorporated herein by reference and in its entirety for all purposes.

Some embodiments may include one or more insight engines to deliver information, characteristics, or determinations related to a disease state that may be based on genetic and/or clinical data associated with a patient, specimen and/or organoid. Exemplary insight engines may include a tumor of unknown origin (tumor origin) engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, a line of therapy engine, a metastatic prediction engine, an 10 progression risk prediction engine, and so forth.

An example tumor origin or tumor of unknown origin engine is disclosed, for example, in U.S. patent application Ser. No. 15/930,234, titled “Systems and Methods for Multi-Label Cancer Classification”, and filed May 12, 1920, which is incorporated herein by reference and in its entirety for all purposes.

An example of an HLA LOH engine is disclosed, for example, in U.S. Pat. No. 11,081,210, titled “Detection of Human Leukocyte Antigen Class I Loss of Heterozygosity in Solid Tumor Types by NGS DNA Sequencing”, and issued Aug. 3, 2021, which is incorporated herein by reference and in its entirety for all purposes. An additional example of an HLA LOH engine is disclosed, for example, in U.S. patent application Ser. No. 17/304,940, titled “Detection of Human Leukocyte Antigen Loss of Heterozygosity”, and filed Jun. 28, 2021, which is incorporated herein by reference and in its entirety for all purposes.

An example of a tumor mutational burden (TMB) engine is disclosed, for example, in U.S. Patent Publication No. 2020/0258601, titled “Targeted-Panel Tumor Mutational Burden Calculation Systems and Methods”, and published Aug. 13, 2020, which is incorporated herein by reference and in its entirety for all purposes.

An example of a PD-L1 status engine is disclosed, for example, in U.S. Patent Publication No. 2020/0395097, titled “A Pan-Cancer Model to Predict The PD-L1 Status of a Cancer Cell Sample Using RNA Expression Data and Other Patient Data”, and published Dec. 17, 2020, which is incorporated herein by reference and in its entirety for all purposes. An additional example of a PD-L1 status engine is disclosed, for example, in U.S. Pat. No. 10,957,041, titled “Determining Biomarkers from Histopathology Slide Images”, issued Mar. 23, 2021, which is incorporated herein by reference and in its entirety for all purposes.

An example of a homologous recombination deficiency engine is disclosed, for example, in U.S. Pat. No. 10,975,445, titled “An Integrative Machine-Learning Framework to Predict Homologous Recombination Deficiency”, and issued Apr. 13, 2021, which is incorporated herein by reference and in its entirety for all purposes. An additional example of a homologous recombination deficiency engine is disclosed, for example, in U.S. patent application Ser. No. 17/492,518, titled “Systems and Methods for Predicting Homologous Recombination Deficiency Status of a Specimen”, filed Oct. 1, 2021, which is incorporated herein by reference and in its entirety for all purposes.

An example of a cellular pathway activation report engine is disclosed, for example, in U.S. Patent Publication No. 2021/0057042, titled “Systems And Methods For Detecting Cellular Pathway Dysregulation In Cancer Specimens”, and published Feb. 25, 2021, which is incorporated herein by reference and in its entirety for all purposes.

An example of an immune infiltration engine is disclosed, for example, in U.S. Patent Publication No. 2020/0075169, titled “A Multi-Modal Approach to Predicting Immune Infiltration Based on Integrated RNA Expression and Imaging Features”, and published Mar. 5, 2020, which is incorporated herein by reference and in its entirety for all purposes.

An example of an MSI engine is disclosed, for example, in U.S. Patent Publication No. 2020/0118644, titled “Microsatellite Instability Determination System and Related Methods”, and published Apr. 16, 2020, which is incorporated herein by reference and in its entirety for all purposes. An additional example of an MSI engine is disclosed, for example, in U.S. Patent Publication No. 2021/0098078, titled “Systems and Methods for Detecting Microsatellite Instability of a Cancer Using a Liquid Biopsy”, and published Apr. 1, 2021, which is incorporated herein by reference and in its entirety for all purposes.

An example of a pathogen infection status engine is disclosed, for example, in U.S. Pat. No. 11,043,304, titled “Systems And Methods For Using Sequencing Data For Pathogen Detection”, and issued Jun. 22, 2021, which is incorporated herein by reference and in its entirety for all purposes. Another example of a pathogen infection status engine is disclosed, for example, in PCT/US21/18619, titled “Systems And Methods For Detecting Viral DNA From Sequencing”, and filed Feb. 18, 2021, which is incorporated herein by reference and in its entirety for all purposes.

An example of a T cell receptor or B cell receptor profiling engine is disclosed, for example, in U.S. patent application Ser. No. 17/302,030, titled “TCR/BCR Profiling Using Enrichment with Pools of Capture Probes”, and filed Apr. 21, 2021, which is incorporated herein by reference and in its entirety for all purposes.

An example of a line of therapy engine is disclosed, for example, in U.S. Patent Publication No. 2021/0057071, titled “Unsupervised Learning And Prediction Of Lines Of Therapy From High-Dimensional Longitudinal Medications Data”, and published Feb. 25, 2021, which is incorporated herein by reference and in its entirety for all purposes.

An example of a metastatic prediction engine is disclosed, for example, in U.S. Pat. No. 11,145,416, titled “Predicting likelihood and site of metastasis from patient records”, and issued Oct. 12, 2021, which is incorporated herein by reference and in its entirety for all purposes.

An example of an IO progression risk prediction engine is disclosed, for example, in U.S. patent application Ser. No. 17/455,876, titled “Determination of Cytotoxic Gene Signature and Associated Systems and Methods For Response Prediction and Treatment”, and filed Nov. 19, 2021, which is incorporated herein by reference and in its entirety for all purposes.

An additional example of a microsatellite instability engine is disclosed, for example, in U.S. patent application Ser. No. 16/412,362, titled “A Generalizable and Interpretable Deep Learning Framework for Predicting MSI From Histopathology Slide Images”, and filed May 14, 2019, which is incorporated herein by reference and in its entirety for all purposes.

An example of a radiomics engine is disclosed, for example, in U.S. patent application Ser. No. 16/460,975, titled “3D Radiomic Platform for Imaging Biomarker Development”, and filed Jul. 2, 2019, which is incorporated herein by reference and in its entirety for all purposes.

An example of a tissue segmentation engine is disclosed, for example, in U.S. patent application Ser. No. 16/732,242, titled “Artificial Intelligence Segmentation Of Tissue Images”, and filed Dec. 31, 2019, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician, including embedding predictions herein (unimodal and multimodal). For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen, as well the as the embedding predictions. The genetic profile may represent genetic sequences present in the tissue type, tumor, or organ and may include variants, expression levels, information about gene products, or other information that could be derived from genetic analysis of a tissue, tumor, or organ.

The report may include therapies and/or clinical trials matched based on a portion or all of the genetic profile or insight engine findings and summaries, including the based on the embedding predictions. For example, the clinical trials may be matched according to the systems and methods disclosed in U.S. Patent Publication No. 2020/0381087, titled “Systems and Methods of Clinical Trial Evaluation”, published Dec. 3, 2020, which is incorporated herein by reference and in its entirety for all purposes.

The report may include a comparison of the results (for example, molecular and/or clinical patient data) to a database of results from many specimens. An example of methods and systems for comparing results to a database of results are disclosed in U.S. Patent Publication No. 2020/0135303 titled “User Interface, System, And Method For Cohort Analysis” and published Apr. 30, 2020, and U.S. Patent Publication No. 2020/0211716 titled “A Method and Process for Predicting and Analyzing Patient Cohort Response, Progression and Survival”, and published Jul. 2, 2020, which is incorporated herein by reference and in its entirety for all purposes. The information may be used, sometimes in conjunction with similar information from additional specimens and/or clinical response information, to match therapies likely to be successful in treating a patient, discover biomarkers or design a clinical trial.

Any data generated by the systems and methods and/or the digital and laboratory health care platform may be downloaded by the user. In one example, the data may be downloaded as a CSV file comprising clinical and/or molecular data associated with tests, data structuring, and/or other services ordered by the user. In various embodiments, this may be accomplished by aggregating clinical data in a system backend, and making it available via a portal. This data may include not only variants and RNA expression data, but also data associated with immunotherapy markers such as MSI and TMB, as well as RNA fusions.

Some embodiments include a device comprising a microphone and speaker for receiving audible queries or instructions from a user and delivering answers or other information, the methods and systems described above may be utilized to add data to a database the device can access. An example of such a device is disclosed, for example, in U.S. Patent Publication No. 2020/0335102, titled “Collaborative Artificial Intelligence Method And System”, and published Oct. 22, 2020, which is incorporated herein by reference and in its entirety for all purposes.

Some embodiments include a mobile application for ingesting patient records, including genomic sequencing records and/or results even if they were not generated by the same digital and laboratory health care platform, the methods and systems described above may be utilized to receive ingested patient records. An example of such a mobile application is disclosed, for example, in U.S. Pat. No. 10,395,772, titled “Mobile Supplementation, Extraction, And Analysis Of Health Records”, and issued Aug. 27, 2019, which is incorporated herein by reference and in its entirety for all purposes. Another example of such a mobile application is disclosed, for example, in U.S. Pat. No. 10,902,952, titled “Mobile Supplementation, Extraction, And Analysis Of Health Records”, and issued Jan. 26, 2021, which is incorporated herein by reference and in its entirety for all purposes. Another example of such a mobile application is disclosed, for example, in U.S. Patent Publication No. 2021/0151192, titled “Mobile Supplementation, Extraction, And Analysis Of Health Records”, and filed May 20, 2021, which is incorporated herein by reference and in its entirety for all purposes.

Some embodiments include organoids developed in connection with the platform (for example, from the patient specimen), the methods and systems may be used to further evaluate genetic sequencing data derived from an organoid and/or the organoid sensitivity, especially to therapies matched based on a portion or all of the information determined by the systems and methods, including predicted cancer type(s), likely tumor origin(s), etc. These therapies may be tested on the organoid, derivatives of that organoid, and/or similar organoids to determine an organoid's sensitivity to those therapies. Any of the results may be included in a report. If the organoid is associated with a patient specimen, any of the results may be included in a report associated with that patient and/or delivered to the patient or patient's physician or clinician. In various examples, organoids may be cultured and tested according to the systems and methods disclosed in U.S. Patent Publication No. 2021/0155989, titled “Tumor Organoid Culture Compositions, Systems, and Methods”, published May 27, 2021; PCT/US20/56930, titled “Systems and Methods for Predicting Therapeutic Sensitivity”, filed Oct. 22, 2020; U.S. Patent Publication No. 2021/0172931, titled “Large Scale Organoid Analysis”, published Jun. 10, 2021; PCT/US2020/063619, titled “Systems and Methods for High Throughput Drug Screening”, filed Dec. 7, 2020 and U.S. patent application Ser. No. 17/301,975, titled “Artificial Fluorescent Image Systems and Methods”, filed Apr. 20, 2021 which are each incorporated herein by reference and in their entirety for all purposes. In one example, the drug sensitivity assays may be especially informative if the systems and methods return results that match with a variety of therapies, or multiple results (for example, multiple equally or similarly likely cancer types or tumor origins), each matching with at least one therapy.

Some embodiments include an application of one or more of the above in combination with or as part of a medical device or a laboratory developed test that is generally targeted to medical care and research, such laboratory developed test or medical device results may be enhanced and personalized through the use of artificial intelligence. In an example, a laboratory test may determine a biomarker, which is in turn used to determine a mental health disease state, or a prediction related to a mental health disease state. For instance, the mental health disease states may include, depression, a mental disorder, a behavioral disorder, a personality disorder, etc. Examples of predictions related to the mental health disease state may include a predicted response to a therapy, a suitability for a therapy, a progression of a mental disease state, a suitability for a clinical trial, etc. In another example, a laboratory test may determine a biomarker, which is in turn used to determine an endocrinological disease state, such as diabetes, thyroidism, an autoimmune disease state. Additionally or alternatively, the biomarker may be used to determine a prediction related to the endocrinological disease state, such as a predicted response to a therapy, a suitability for a therapy, a progression of a disease state, and a suitability for a clinical trial. In yet another example, a laboratory test may determine a biomarker, which is in turn used to determine a cardiovascular disease state, such as arrhythmia, cardiac arrest, stroke, atrial fibrillation, aortic stenosis, amyloidosis, etc. Additionally or alternatively, the biomarker may be used to determine a prediction related to the cardiovascular disease, such as a response to a therapy, a suitability for a therapy, a progression of a cardiovascular disease state, and a suitability for a clinical trial. An example of laboratory developed tests, especially those that may be enhanced by artificial intelligence, is disclosed, for example, in U.S. Patent Publication No. 2021/0118559, titled “Artificial Intelligence Assisted Precision Medicine Enhancements to Standardized Laboratory Diagnostic Testing”, and published Apr. 22, 2021, which is incorporated herein by reference and in its entirety for all purposes.

It should be understood that the examples given above are illustrative and do not limit the uses of the systems and methods described herein in combination with a digital and laboratory health care platform.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

### ADDITIONAL EXEMPLARY EMBODIMENTS

Aspect 1. A method for transforming a plurality of nucleic acid reads to one or more genomic biomarkers, the method performed by one or more processors, the method comprising:
receiving, from a data source, an order to transform the plurality of nucleic acid reads to the one or more genomic biomarkers, wherein the plurality of nucleic acid reads are derived from next generation sequencing of a specimen;
selecting a transform for the order, wherein the transform comprises a configuration, a transform image comprising a plurality of indications of storage locations and a plurality of instructions for completing the transform;
associating the selected transform with a cloud computing platform based at least in part on the configuration, the association comprising:
   providing, to the cloud computing platform, the transform image;
   executing, via the cloud computing platform, the plurality of instructions for completing the selected transform; and
   loading, via a communication interface, the plurality of nucleic acid reads into a first storage location indicated by the plurality of indications of storage locations;
communicating, via the communication interface, communications from the execution between the selected transform and the data source, the communications comprising at least an operational status of the selected transform;
storing, via the communication interface, the genomic biomarker output from the selected transform into a second storage location indicated by the plurality of indications of storage locations; and
providing a notification, to the data source via the communication interface, of a final operational status of the selected transform based at least in part on the storing the genomic biomarker output from the selected transform.

Aspect 2. The method of aspect 1, wherein the one or more genomic biomarkers are selected from:
a microsatellite instability,
a tumor mutational burden,
a variant characterization,
a copy number variation,
a fusion, and
a presence of a stain image-derived biomarker.

Aspect 3. The method of any one of aspects 1-2, wherein the genomic biomarker comprises the MSI, the method further comprising:
comparing regions of the genome to at least a portion of the plurality of nucleic acid reads to identify differences and similarities; and
reporting the MSI, wherein the MSI comprises a ratio of the identified differences to similarities.

Aspect 4. The method of any one of aspects 1-3, further comprising:
accessing, with the transform, an input directory, wherein the input directory is separate from the data source; and
writing, with the transform, to an output directory, wherein the output directory is separate from the data source.

Aspect 5. The method of any one of aspects 1-4, wherein the plurality of nucleic acid reads are in a FASTQ format or a BAMF format.

Aspect 6. The method of any one of aspects 1-5, wherein the plurality of nucleic acid reads are aligned to a common reference genome.

Aspect 7. The method of any one of aspects 1-6, wherein the transforms are associated with the cloud computing platforms based on compute requirements of the order to transform the plurality of nucleic acid reads.

Aspect 8. The method of any one of aspects 1-7, wherein:
the transforms are associated with the cloud computing platforms based on an available virtual machine (VM) memory size, an available central processing unit (CPU) performance, and a resource quota; and
the resource quota comprises a constraint on total compute resources available to: (i) a group of transforms, (ii) a cloud computing system, and/or, (iii) a portion of a cloud computing system.

Aspect 9. The method of any one of aspects 1-8, further comprising:
in response to receiving the order to transform the plurality of nucleic acid reads to the one or more genomic biomarkers, determining if the plurality of nucleic acid reads are available to be read; and
wherein the selecting of the transforms occurs in response to a determination that the plurality of nucleic acid reads are available to be read.

Aspect 10. The method of any one of aspects 1-9, further comprising:
creating a catalog of transforms for deriving genomic biomarkers by:
   receiving a plurality of transforms for deriving genomic biomarkers;
   validating each transform of the received plurality of transforms by determining if there is a problem with each transform;
   if there is a problem with a transform, returning an error message including an indication of the problem; and
   updating at least one transform of the plurality of transforms by:
      receiving an update to the at least one transform;
      validating the at least one transform by determining if there is a problem with the at least one transform; and
      if there is a problem with the at least one transform, returning an error message including an indication of the problem with the at least one transform; and
wherein the selecting of the transforms comprises selecting the transforms from the created catalog of transforms.

Aspect 11. The method of any one of aspects 1-10, wherein the notification of the operational status of the data source includes an orchestration error, an execution error, or a timeout error.

Aspect 12. The method of any one of aspects 1-11, further comprising:
upon receiving the order to transform the plurality of nucleic acid reads to the one or more genomic biomarkers, determining if the transform should be placed in a high priority queue or a low priority queue; and
depending on the determination, placing the order in either the high priority queue or low priority queue; and
wherein the executing the plurality of instructions for completing the selected transform occurs by executing instructions in the high priority queue before executing instructions in the low priority queue.

Aspect 13. The method of any one of aspects 1-12, wherein the configuration is cloud computing platform agnostic.

Aspect 14. The method of any one of aspects 1-13, further comprising predicting, based on the stored genomic biomarker, a likelihood of a patient being at a high-risk of one or more of an oncological event, neurological disorder, autoimmune condition, cardiovascular disease, infectious disease, or endocrinological disease.

Aspect 15. The method any one of aspects 1-14, further comprising predicting, based on the stored genomic biomarker, one or more of:
an onset of an oncological disease state;
an onset of cancer;
a response to a cancer therapy;
a suitability for a cancer therapy;
a suitability for a cancer clinical trial;
a progression free cancer survival;
a progression of cancer;
a metastasis of cancer; and/or
an origin of a metastasized tumor.

16. The method of any one of aspects 1-15, further comprising predicting, based on the stored genomic biomarker, one or more of:
an onset of an endocrinological disease state;
an onset of diabetes;
an onset of thyroidism;
an onset of an autoimmune disease state;
a response to an endocrinological therapy;
a suitability for an endocrinological therapy;
a progression of an endocrinological disease state; and/or
a suitability for an endocrinological clinical trial.

17. The method of any one of aspects 1-16, further comprising predicting, based on the stored genomic biomarker, one or more of:
an onset of a mental health disease state;
an onset of depression;
an onset of a mental disorder;
an onset of a behavioral disorder;
an onset of a personality disorder;
a response to a neurological therapy;
a suitability for a neurological therapy;
a progression of a mental health disease state; and/or
a suitability for a neurological clinical trial.

18. The method any one of aspects 1-17, further comprising predicting, based on the stored genomic biomarker, one or more of:
an onset of a cardiovascular disease state;
an onset of an arrhythmia;
an onset of cardiac arrest;
an onset of stroke;
an onset of atrial fibrillation;
an onset of aortic stenosis;
an onset of amyloidosis;
a response to a cardiovascular therapy;
a suitability for a cardiovascular therapy;
a progression of a cardiovascular disease state; and/or
a suitability for a cardiovascular clinical trial.

Aspect 19. A computer system for transforming a plurality of nucleic acid reads to one or more genomic biomarkers, the computer system comprising one or more processors configured to:
receive, from a data source, an order to transform the plurality of nucleic acid reads to the one or more genomic biomarkers, wherein the plurality of nucleic acid reads are derived from next generation sequencing of a specimen;
select a transform for the order, wherein the transform comprises a configuration, a transform image comprising a plurality of indications of storage locations and a plurality of instructions for completing the transform;
associate the selected transform with a cloud computing platform based at least in part on the configuration, the association comprising:
   providing, to the cloud computing platform, the transform image;
   executing, via the cloud computing platform, the plurality of instructions for completing the selected transform; and
   loading, via a communication interface, the plurality of nucleic acid reads into a first storage location indicated by the plurality of indications of storage locations;
communicate, via the communication interface, communications from the execution between the selected transform and the data source, the communications comprising at least an operational status of the selected transform;
store, via the communication interface, the genomic biomarker output from the selected transform into a second storage location indicated by the plurality of indications of storage locations; and
provide a notification, to the data source via the communication interface, a final operational status of the selected transform based at least in part on the storing the genomic biomarker output from the selected transform.

Aspect 20. A computing device for transforming a plurality of nucleic acid reads to one or more genomic biomarkers, the computing device comprising:
one or more processors; and
one or more memories coupled to the one or more processors;
the one or more memories including computer executable instructions stored therein that, when executed by the one or more processors, cause the one or more processors to:
select a transform for the order, wherein the transform comprises a configuration, a transform image comprising a plurality of indications of storage locations and a plurality of instructions for completing the transform;
associate the selected transform with a cloud computing platform based at least in part on the configuration, the association comprising:
   providing, to the cloud computing platform, the transform image;
   executing, via the cloud computing platform, the plurality of instructions for completing the selected transform; and
   loading, via a communication interface, the plurality of nucleic acid reads into a first storage location indicated by the plurality of indications of storage locations;
communicate, via the communication interface, communications from the execution between the selected transform and the data source, the communications comprising at least an operational status of the selected transform;
store, via the communication interface, the genomic biomarker output from each selected transform into a second storage location indicated by the plurality of indications of storage locations; and
provide a notification, to the data source via the communication interface, a final operational status of the selected transform based at least in part on the storing the genomic biomarker output from the selected transform.

### ADDITIONAL ALTERNATIVE EMBODIMENTS

Embodiment 1. A method for transforming biological data to one or more genomic biomarkers, the method performed by one or more processors, the method comprising:
receiving, from a data source, an order to transform the biological data to the one or more genomic biomarkers;
selecting a transform for the order, wherein the transform comprises a configuration, a transform image comprising a plurality of indications of storage locations and a plurality of instructions for completing the transform;
associating the selected transform with a cloud computing platform based at least in part on the configuration, the association comprising:
   providing, to the cloud computing platform, the transform image;
   executing, via the cloud computing platform, the plurality of instructions for completing the selected transform; and
   loading, via a communication interface, the biological data into a first storage location indicated by the plurality of indications of storage locations;
communicating, via the communication interface, communications from the execution between the selected transform and the data source, the communications comprising at least an operational status of the selected transform;
storing, via the communication interface, the genomic biomarker output from the selected transform into a second storage location indicated by the plurality of indications of storage locations; and
providing a notification, to the data source via the communication interface, of a final operational status of the selected transform based at least in part on the storing the genomic biomarker output from the selected transform.

Embodiment 2. The method of embodiment 1, wherein the biological data includes slide images, H&E stain images, IHC images, and/or radiology images.

Embodiment 3. The method of any one of embodiments 1-2, further comprising:
accessing, with the transform, an input directory, wherein the input directory is separate from the data source; and
writing, with the transform, to an output directory, wherein the output directory is separate from the data source.

Embodiment 4. The method of any one of embodiments 1-3, wherein the transforms are associated with the cloud computing platforms based on compute requirements of the order to transform the biological data.

Embodiment 5. The method of any one of embodiments 1-4, wherein:
the transforms are associated with the cloud computing platforms based on an available virtual machine (VM) memory size, an available central processing unit (CPU) performance, and a resource quota; and
the resource quota comprises a constraint on total compute resources available to: (i) a group of transforms, (ii) a cloud computing system, and/or, (iii) a portion of a cloud computing system.

Embodiment 6. The method of any one of embodiments 1-5, further comprising:
in response to receiving the order to transform the biological data to the one or more genomic biomarkers, determining if the biological data is available to be read; and
wherein the selecting of the transforms occurs in response to a determination that the biological data is available to be read.

Embodiment 7. The method of any one of embodiments 1-6, further comprising:
creating a catalog of transforms for deriving genomic biomarkers by:
   receiving a plurality of transforms for deriving genomic biomarkers;
   validating each transform of the received plurality of transforms by determining if there is a problem with each transform;
   if there is a problem with a transform, returning an error message including an indication of the problem; and
   updating at least one transform of the plurality of transforms by:
      receiving an update to the at least one transform;
      validating the at least one transform by determining if there is a problem with the at least one transform; and
      if there is a problem with the at least one transform, returning an error message including an indication of the problem with the at least one transform; and
wherein the selecting of the transforms comprises selecting the transforms from the created catalog of transforms.

Embodiment 8. The method of any one of embodiments 1-7, wherein the notification of the operational status of the data source includes an orchestration error, an execution error, or a timeout error.

Embodiment 9. The method of any one of embodiments 1-8, further comprising:
upon receiving the order to transform the biological data to the one or more genomic biomarkers, determining if the transform should be placed in a high priority queue or a low priority queue; and
depending on the determination, placing the order in either the high priority queue or low priority queue; and
wherein the executing the plurality of instructions for completing the selected transform occurs by executing instructions in the high priority queue before executing instructions in the low priority queue.

Embodiment 10. The method of any one of embodiments 1-9, wherein the configuration is cloud computing platform agnostic.

Embodiment 11. The method of any one of embodiments 1-10, further comprising predicting, based on the stored genomic biomarker, a likelihood of a patient being at a high-risk of one or more of an oncological event, neurological disorder, autoimmune condition, cardiovascular disease, infectious disease, or endocrinological disease.

Embodiment 12. A computer system for transforming biological data to one or more genomic biomarkers, the computer system comprising one or more processors configured to:
receive, from a data source, an order to transform the biological data to the one or more genomic biomarkers;
select a transform for the order, wherein the transform comprises a configuration, a transform image comprising a plurality of indications of storage locations and a plurality of instructions for completing the transform;
associate the selected transform with a cloud computing platform based at least in part on the configuration, the association comprising:
   providing, to the cloud computing platform, the transform image;
   executing, via the cloud computing platform, the plurality of instructions for completing the selected transform; and
   loading, via a communication interface, the biological data into a first storage location indicated by the plurality of indications of storage locations;
communicate, via the communication interface, communications from the execution between the selected transform and the data source, the communications comprising at least an operational status of the selected transform;
store, via the communication interface, the genomic biomarker output from the selected transform into a second storage location indicated by the plurality of indications of storage locations; and
provide a notification, to the data source via the communication interface, a final operational status of the selected transform based at least in part on the storing the genomic biomarker output from the selected transform.

What is claimed:

1. A method for transforming a plurality of nucleic acid reads to one or more genomic biomarkers, the method performed by one or more processors, the method comprising:
receiving, from a data source, an order to transform the plurality of nucleic acid reads to the one or more genomic biomarkers, wherein the plurality of nucleic acid reads are derived from next generation sequencing of a specimen;
receiving a selection of a transform for the order, wherein the transform comprises a configuration, a transform image comprising a plurality of indications of storage locations and a plurality of instructions for completing the transform;
associating the selected transform with a cloud computing platform based at least in part on the configuration, the association comprising:
providing, to the cloud computing platform, the transform image;
executing, via the cloud computing platform, the plurality of instructions for completing the selected transform; and
loading, via a communication interface, the plurality of nucleic acid reads into a first storage location indicated by the plurality of indications of storage locations;
communicating, via the communication interface, at least one communication from the execution between the selected transform and the data source, the at least one communication comprising at least an operational status of the selected transform;
storing, via the communication interface, the genomic biomarker output from the selected transform into a second storage location indicated by the plurality of indications of storage locations; and
providing a notification, to the data source via the communication interface, of a final operational status of the selected transform based at least in part on the storing the genomic biomarker output from the selected transform.

2. The method of claim 1, wherein the one or more genomic biomarkers are selected from:
a microsatellite instability (MSI),
a tumor mutational burden,
a variant characterization,
a copy number variation,
a fusion, and
a presence of a stain image-derived biomarker.

3. The method of claim 2, wherein the genomic biomarker comprises the MSI, the method further comprising:
comparing regions of the genome to at least a portion of the plurality of nucleic acid reads to identify differences and similarities; and
reporting the MSI, wherein the MSI comprises a ratio of the identified differences to similarities.

4. The method of claim 1, further comprising:
accessing, with the transform, an input directory, wherein the input directory is separate from the data source; and
writing, with the transform, to an output directory, wherein the output directory is separate from the data source.

5. The method of claim 1, wherein the plurality of nucleic acid reads are in a FASTQ format or a BAMF format.

6. The method of claim 1, wherein the plurality of nucleic acid reads are aligned to a common reference genome.

7. The method of claim 1, wherein the transforms are associated with the cloud computing platforms based on compute requirements of the order to transform the plurality of nucleic acid reads.

8. The method of claim 1, wherein:
the transforms are associated with the cloud computing platforms based on an available virtual machine (VM) memory size, an available central processing unit (CPU) performance, and a resource quota; and
the resource quota comprises a constraint on total compute resources available to: (i) a group of transforms, (ii) a cloud computing system, and/or, (iii) a portion of a cloud computing system.

9. The method of claim 1, further comprising:
in response to receiving the order to transform the plurality of nucleic acid reads to the one or more genomic biomarkers, determining if the plurality of nucleic acid reads are available to be read; and
wherein the selecting of the transforms occurs in response to a determination that the plurality of nucleic acid reads are in available to be read.

10. The method of claim 1, further comprising:
creating a catalog of transforms for deriving genomic biomarkers by:
receiving a plurality of transforms for deriving genomic biomarkers;
validating each transform of the received plurality of transforms by determining if there is a problem with each transform;
if there is a problem with a transform, returning an error message including an indication of the problem; and
updating at least one transform of the plurality of transforms by:
receiving an update to the at least one transform;
validating the at least one transform by determining if there is a problem with the at least one transform; and
if there is a problem with the at least one transform, returning an error message including an indication of the problem with the at least one transform; and
wherein the selecting of the transforms comprises selecting the transforms from the created catalog of transforms.

11. The method of claim 1, wherein the notification of the operational status of the data source includes an orchestration error, an execution error, or a timeout error.

12. The method of claim 1, further comprising:
upon receiving the order to transform the plurality of nucleic acid reads to the one or more genomic biomarkers, determining if the transform should be placed in a high priority queue or a low priority queue; and
depending on the determination, placing the order in either the high priority queue or low priority queue; and
wherein the executing the plurality of instructions for completing the selected transform occurs by executing instructions in the high priority queue before executing instructions in the low priority queue.

13. The method of claim 1, wherein the configuration is cloud computing platform agnostic.

14. The method of claim 1, further comprising predicting, based on the stored genomic biomarker, a likelihood of a patient being at a high-risk of one or more of an oncological event, neurological disorder, autoimmune condition, cardiovascular disease, infectious disease, or endocrinological disease.

15. The method of claim 1, further comprising predicting, based on the stored genomic biomarker, one or more of:
an onset of an oncological disease state;
an onset of cancer;
a response to a cancer therapy;
a suitability for a cancer therapy;
a suitability for a cancer clinical trial;
a progression free cancer survival;
a progression of cancer;
a metastasis of cancer; and/or
an origin of a metastasized tumor.

16. The method of claim 1, further comprising predicting, based on the stored genomic biomarker, one or more of:
an onset of an endocrinological disease state;
an onset of diabetes;
an onset of thyroidism;
an onset of an autoimmune disease state;
a response to an endocrinological therapy;
a suitability for an endocrinological therapy;
a progression of an endocrinological disease state; and/or
a suitability for an endocrinological clinical trial.

17. The method of claim 1, further comprising predicting, based on the stored genomic biomarker, one or more of:
an onset of a mental health disease state;
an onset of depression;
an onset of a mental disorder;
an onset of a behavioral disorder;
an onset of a personality disorder;
a response to a neurological therapy;
a suitability for a neurological therapy;
a progression of a mental health disease state; and/or
a suitability for a neurological clinical trial.

18. The method of claim 1, further comprising predicting, based on the stored genomic biomarker, one or more of:
an onset of a cardiovascular disease state;
an onset of an arrhythmia;
an onset of cardiac arrest;
an onset of stroke;
an onset of atrial fibrillation;
an onset of aortic stenosis;
an onset of amyloidosis;
a response to a cardiovascular therapy;
a suitability for a cardiovascular therapy;
a progression of a cardiovascular disease state; and/or
a suitability for a cardiovascular clinical trial.

19. A computer system for transforming a plurality of nucleic acid reads to one or more genomic biomarkers, the computer system comprising one or more processors configured to:
receive, from a data source, an order to transform the plurality of nucleic acid reads to the one or more genomic biomarkers, wherein the plurality of nucleic acid reads are derived from next generation sequencing of a specimen;
receive a selection of a transform for the order, wherein the transform comprises a configuration, a transform image comprising a plurality of indications of storage locations and a plurality of instructions for completing the transform;
associate the selected transform with a cloud computing platform based at least in part on the configuration, the association comprising:
providing, to the cloud computing platform, the transform image;
executing, via the cloud computing platform, the plurality of instructions for completing the selected transform; and
loading, via a communication interface, the plurality of nucleic acid reads into a first storage location indicated by the plurality of indications of storage locations;
communicate, via the communication interface, at least one communication from the execution between the selected transform and the data source, the at least one communication comprising at least an operational status of the selected transform;
store, via the communication interface, the genomic biomarker output from the selected transform into a second storage location indicated by the plurality of indications of storage locations; and
provide a notification, to the data source via the communication interface, a final operational status of the selected transform based at least in part on the storing the genomic biomarker output from the selected transform.

20. A computing device for transforming plurality of nucleic acid reads to one or more genomic biomarkers, the computing device comprising:
one or more processors; and
one or more memories coupled to the one or more processors;
the one or more memories including computer executable instructions stored therein that, when executed by the one or more processors, cause the one or more processors to:
receive, from a data source, an order to transform the plurality of nucleic acid reads to the one or more genomic biomarkers, wherein the plurality of nucleic acid reads are derived from next generation sequencing of a specimen;
receive a selection of a transform for the order, wherein the transform comprises a configuration, a transform image comprising a plurality of indications of storage locations and a plurality of instructions for completing the transform;
associate the selected transform with a cloud computing platform based at least in part on the configuration, the association comprising:
providing, to the cloud computing platform, the transform image;
executing, via the cloud computing platform, the plurality of instructions for completing the selected transform; and
loading, via a communication interface, the plurality of nucleic acid reads into a first storage location indicated by the plurality of indications of storage locations;
communicate, via the communication interface, at least one communication from the execution between the selected transform and the data source, the at least one communication comprising at least an operational status of the selected transform;
store, via the communication interface, the genomic biomarker output from the selected transform into a second storage location indicated by the plurality of indications of storage locations; and
provide a notification, to the data source via the communication interface, a final operational status of the selected transform based at least in part on the storing the genomic biomarker output from the selected transform.
